(19) United States
(12) Patent Application Publication
(10) Pub. No.: US 2026/0224739 A1
(43) Pub. Date: Aug. 6, 2026

(54) Fluorescent Emission Spectral Stacking of Polymer Encapsulated Quantum Dots

(71) Applicant: Core Quantum Technologies, Inc., Columbus, OH (US)

(72) Inventors: Kristie Marie Krug, Upper Arlington, OH (US); Mythreyi Unni, Powell, OH (US)

(73) Assignee: Core Quantum Technologies, Inc., Columbus, OH (US)

(21) Appl. No.: 19/578,728

(22) Filed: Mar. 25, 2026

Related U.S. Application Data
(63) Continuation-in-part of application No. 19/251,037, filed on Jun. 26, 2025.
Application No. 19/251,037 is a continuation of application No. 17/943,788, filed on Sep. 13, 2022, now Pat. No. 12,370,266.

(51) Int. Cl. A61K 47/69; A61K 47/68; B82Y 15/00; G01N 33/543; G01N 33/569

(52) U.S. Cl. CPC A61K 47/6931; A61K 47/6883; B82Y 15/00; G01N 33/5434; G01N 33/56972

(57) ABSTRACT

Generally, a polymer nanomaterial encapsulation system useful in the production of polymer encapsulated nanoparticles comprised of a hydrophobic nanoparticle encapsulated in the hydrophobic region of the polymer with the external hydrophilic region of the polymer ensuring water-solubility and affording a functional group which can be utilized for the production of nanoparticle conjugates. Specifically, particular embodiments include a polymer nanoparticle structure including quantum dots having narrow spectral emission with emission peaks separated by about 20 nm in the visible spectrum of about 380 nm to about 700 nm and emission peaks separated by about 50 nm in the visible spectrum of about 700 nm to about 1000 nm for the production of antibody conjugates useful in the capture and analysis of cellular targets by spectral flow cytometery.

### I. FIELD OF THE INVENTION

Generally, a nanomaterial polymer encapsulation system useful in the production of nanocomposites comprising a hydrophobic nanoparticle encapsulated in a hydrophobic region of a polymer with the external hydrophilic region of the polymer ensuring water-solubility and affording a functional group which can be utilized for the production of nanocomposite conjugates.

Specifically, particular embodiments can comprise nanocomposites including one or more nanoparticles including a quantum dot (“QD”) and/or a superparamagnetic iron oxide nanoparticle (“SPION”) and/or an upconverting nanoparticle encapsulated in a polystyrene-b-polyethylene glycol amine (“PS-b-PEG-NH₂”) affording an amine functional group that can be activated to conjugate antibodies, modified antibodies, or antibody fragments, and in a particular embodiments, activated with methyltetrazine polyethylene glycol-4-N-hydroxysuccinimide esters (“methyltetrazine-PEG4-NHS ester”) to allow conjugation of trans-cyclooctyne (“TCO”) modified antibody or antibody fragments.

In particular embodiments, nanocomposite sets or quantum dot sets including quantum dots having narrow spectral emission with emission peaks measured at full width at half maximum separated by about 20 nm in the visible spectrum of about 380 nm to about 700 nm and emission peaks measured at full width at half maximum separated by about 50 nm in the visible spectrum of about 700 nm to about 1000 nm.

### II. BACKGROUND OF THE INVENTION

Nanocomposites have a diverse variety of potential applications including, but not limited to, medicine, biomedicine, biotechnology, biomaterials, biomechanics, and energy production. Current methods of polymer encapsulation of nanoparticles such as QD or SPION to produce nanocomposites have led to agglomeration of the nanocomposites limiting production to small batches, low photochemical stability, lack of uniform size and brightness, a lack of specificity of nanocomposite conjugates to cellular targets, and the lack of methodologies for rapid purification of nanocomposites bound to cellular targets. Additionally, conventional quantum dots may lack photochemical stability in harsh environments, including biological reagents, and demonstrate decreased quantum yield after conjugation and diminished long-term stability fluorescently and colloidally. Additionally, the co-excitation of many lasers can complicate incorporation of quantum dots in conventional flow cytometry panels in combination with other fluorophores.

There would be substantial advantages in nanocomposites and methods of making and using nanocomposites that minimize agglomeration in large batch production, have high photochemical stability, uniform and narrow size distribution and brightness, high binding specificity to cellular targets, along with protocols for rapid purification of nanocomposites bound to cellular targets, and which allow encapsulation of quantum dots having narrow spectral emission with emission peaks separated by about 20 nm in the visible spectrum of about 380 nm to about 700 nm and emission peaks separated by about 50 nm in the visible spectrum of about 700 nm to about 1000 nm that can be used in spectral flow cytometry and spectral imaging platforms.

### III. SUMMARY OF THE INVENTION

A broad object of particular embodiments of the invention can be to provide a nanocomposite comprising one or more nanoparticles encapsulated by a polymer having a hydrophobic region associated with the nanoparticle and a hydrophilic region including a functional group associated with the aqueous environment, wherein the nanoparticle can comprise one or more of a QD or a SPION, and combinations thereof, and the polymer can comprise a polystyrene-b-polyethylene glycol including a functional group, wherein polystyrene can have a molecular weight occurring in the range of about 1.5 kDa to about 40 kDa, and wherein the polyethylene glycol can have a molecular weight occurring in the range of about 10 kDa to about 40 kDa, whereby combinations and permutations of the QD, SPION, molecular weight of the polystyrene and/or the molecular weight of the polyethylene glycol and selection of the branched structure of the polyethylene glycol, and variation in mass ratios thereof, allow for a numerous and wide variety of nanocomposites to be produced having substantially uniform hydrodynamic diameter occurring in a range of about 40 nm to about 500 nm and brightness due to QD having different emission wavelengths occurring in the range of 420 nm to 1000 nm, and in particular embodiments, can be provided as ordered set of quantum dots having narrow spectral emission with emission peaks separated by about 20 nm in the visible spectrum of about 380 nm to about 700 nm and with emission peaks separated by about 50 nm in the visible spectrum of about 700 nm to about 1000 nm.

Another broad object of particular embodiments of the invention can be to provide a QD and/or SPION nanocomposite antibody conjugate capable of specifically binding a cellular target, wherein illustrative embodiments include nanocomposite antibody conjugates capable of binding lymphocytes, including as representative examples, human lymphocytes having surface cluster of differentiation markers (“CD”), CD3, CD4, CD8, and CD19.

Another broad object of particular embodiments of the invention can be to provide nanocomposites comprising polymer encapsulated QD or SPION, or combinations thereof, for specific targeting of macrophages, wherein illustrative embodiments comprise the uptake of embodiments of nanocomposites by hemocytes.

Another broad object of particular embodiments of the invention can be to provide a method of isolating QD and/or SPION nanocomposite antibody conjugates bound to cells, wherein SPION nanocomposite antibody conjugates bound to the cells can be separated and isolated by influence of a magnetic field, and wherein isolated QD or SPION nanocomposite antibody conjugates bound to cells can be analyzed by flow cytometry, and in particular embodiments the analyzed QD or SPION nanocomposite antibody conjugates bound to the cells can be flow sorted into discrete populations based on one or more characteristics of the cells.

Naturally, further objects of the invention are disclosed throughout other areas of the specification, drawings, photographs, and claims.

### IV. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block flow diagram illustrating in general overview the process to make and use elements of the nanoparticle polymer encapsulation system including: polymer synthesis, preparation of polymer encapsulated nanoparticles, antibody preparation, production of polymer encapsulated nanoparticle conjugated antibodies, and polymer encapsulated nanoparticle conjugated antibodies bound to cellular targets.

FIG. 2 is a block flow diagram including Blocks 2A through 2I illustrating a process to synthesize polymers encompassed by the method of FIG. 1, Block 1A including the illustrative example of PS-b-PEG-NH₂.

FIG. 3 is an NMR spectrum which validates the molecular structure of the illustrative example of PS-b-PEG-NH₂ obtained by the polymer synthesis process shown in FIG. 2, Blocks 2A through 2I.

FIG. 4A illustrates an embodiment of polymer encapsulated nanoparticle encompassed by FIG. 1, Block 1B including one more QD encapsulated in a polymer obtained by the polymer synthesis process shown in FIG. 2, Blocks 2A through 2I.

FIG. 4B illustrates an embodiment of polymer encapsulated nanoparticle encompassed by FIG. 1, Block 1B including one more QD and one or more SPION encapsulated in a polymer obtained by the polymer synthesis process shown in FIG. 2, Blocks 2A through 2I.

FIG. 4C illustrates an embodiment of polymer encapsulated nanoparticle encompassed by FIG. 1, Block 1B including one more QD and/or one or more SPION encapsulated in a polymer obtained by the polymer synthesis process shown in FIG. 2, Blocks 2A through 2I, wherein the polymer can have a functional group which can be activated to bind one or more agents including as illustrative examples: one or more linkers, a polyethylene glycol, a fluorescent probe, an aptamer, a vitamin, a cell surface receptor, a cell coat, a protein, a peptide, a radioactive isotope, a contrast media, a surface charge modifier, a lectin, or, an antibody, a half antibody, an antibody fragment, and combinations thereof.

FIG. 5 is block diagram illustrating a method of assembling the polymer encapsulated nanoparticles shown in FIGS. 4A and 4B by use of electrohydrodynamic mixing mediated-nanoprecipitation.

FIG. 6 is a plot of emissions in the visible region from about 350 nm to about 750 nm of polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots” abbreviated in the legend as “MD peak emission nm”) including emission plots of MD420, MD440, MD450, MD470, MD520, MD580, MD610, MD640, and MD655.

FIG. 7 is a plot of emissions in the near infrared region from about 650 nm to about 1050 nm of polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots” abbreviated in the legend “MD peak emission nm”) including emission plots of MD700, MD800, MD8890, MD900, MD920, and MD1000.

FIG. 8 is a plot of emissions comparing polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots” abbreviated in the legend “MD peak emission nm”) including MD420 and MD655 to Brilliant Violet 421 and Brilliant Violet 650.

FIG. 9A is a micrograph obtained by transmission electron microscopy of a polymer encapsulated nanoparticle encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 using 0.5 v/v of QD in the organic phase.

FIG. 9B is micrograph obtained by transmission electron microscopy of a polymer encapsulated nanoparticle encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 using 0.2 v/v of QD in organic phase.

FIG. 10A is micrograph obtained by transmission electron microscopy of a polymer encapsulated nanoparticle encompassed by FIG. 1, Block 1B including one or more QD having an emission wavelength of 420 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 10B is micrograph obtained by transmission electron microscopy of a polymer encapsulated nanoparticle encompassed by FIG. 1, Block 1B including one or more QD having an emission wavelength of 575 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 10C is micrograph obtained by transmission electron microscopy of a polymer encapsulated nanoparticle encompassed by FIG. 1, Block 1B including one or more QD having an emission wavelength of 610 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 10D is micrograph obtained by transmission electron microscopy of a polymer encapsulated nanoparticle encompassed by FIG. 1, Block 1B including one or more QD having an emission wavelength of 655 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 11A is micrograph obtained by transmission electron microscopy of a polymer encapsulated nanoparticle encompassed by FIG. 1, Block 1B including one or more 20 nm SPION and one or more QD having an emission wavelength occurring at 610 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 11B is micrograph obtained by transmission electron microscopy of a polymer encapsulated nanoparticle encompassed by FIG. 1, Block 1B including one or more 15 nm SPION and one or more QD having an emission wavelength occurring at 610 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 11C is micrograph obtained by transmission electron microscopy of a polymer encapsulated nanoparticle encompassed by FIG. 1, Block 1B including one or more 5 nm SPION and one or more QD having an emission wavelength occurring at 610 nm encapsulated in a polymer obtained by the polymer synthesis process shown in FIG. 2, Blocks 2A through 2I by use of electrohydrodynamic mixing mediated-nanoprecipitation as illustrated in FIG. 5.

FIG. 12A is micrograph obtained by transmission electron microscopy of a polymer encapsulated nanoparticle encompassed by FIG. 1, Block 1B including a ratio of QD:15 nm SPION:polymer (5:5:20) obtained by the polymer synthesis process shown in FIG. 2, Blocks 2A through 2I and by use of electrohydrodynamic mixing mediated-nanoprecipitation as illustrated in FIG. 5.

FIG. 12B is micrograph obtained by transmission electron microscopy of a polymer encapsulated nanoparticle encompassed by FIG. 1, Block 1B including a ratio of QD:5 nm SPION:polymer (3:5:20) obtained by the polymer synthesis process shown in FIG. 2, Blocks 2A through 2I and by use of electrohydrodynamic mixing mediated-nanoprecipitation as illustrated in FIG. 5 and having a hydrodynamic diameter of 210±30 nm.

FIG. 12C is a plot of hydrodynamic diameter against concentration of particles per milliliter of the polymer encapsulated nanoparticles shown in FIG. 9A evidencing a hydrodynamic diameter of about 128 nm±22 nm.

FIG. 12D is a bar graph comparing the iron concentration of polymer encapsulated nanoparticle including SPION of 15 nm and QD having an emission wavelength occurring at 610 nm to polymer encapsulated nanoparticle including SPION of 5 nm and QD having an emission wavelength occurring at 610 nm.

FIG. 12E is a bar graph comparing the fluorescence of polymer encapsulated nanoparticle including SPION of 15 nm to polymer encapsulated nanoparticle including SPION of 5 nm.

FIG. 13A is a flow cytometry univariant histogram depicting detection of peripheral blood mononuclear cells bound to mouse anti human CD4 linked to a polymer encapsulated nanoparticle encompassed by FIG. 1, Block 1D including one or more QD.

FIG. 13B is a flow cytometry univariant histogram depicting detection of peripheral blood mononuclear cells bound to mouse anti human CD3 linked to a polymer encapsulated nanoparticle encompassed by FIG. 1, Block 1D including one or more QD.

FIG. 14A is flow cytometry bivariant dot plot depicting detection of CD3 positive peripheral blood mononuclear cells bound to mouse anti human CD3 linked to a polymer encapsulated nanoparticle encompassed by FIG. 1, Block 1D including one or more SPION and one or more QD and CD3 negative cells in a reaction solution prior to magnetic separation.

FIG. 14B is flow cytometry bivariant dot plot depicting detection of depleted CD3 positive peripheral blood mononuclear cells in the non-magnetic fraction of the reaction solution subsequent to magnetic separation.

FIG. 14C is flow cytometry bivariant dot plot depicting detection of the enriched CD3 positive peripheral blood mononuclear cells bound to mouse anti human CD3 linked to a polymer encapsulated nanoparticle encompassed by FIG. 1, Block 1D including one or more SPION and one or more QD in the magnetic fraction of the reaction solution subsequent to magnetic separation.

FIG. 15A is a brightfield image of host squid hemocytes incubated with inventive nanocomposites including one or more SPION and one or more QD having an emission wavelength occurring at 610 nm.

FIG. 15B is an epifluorescence image of host squid hemocytes incubated with inventive nanocomposites including one or more SPION and one or more QD having an emission wavelength occurring at 610 nm emission detected by fluorescence microscopy in the fluorescein isothiocyanate (“FITC”) channel.

FIG. 15C is composite image of the brightfield and epifluorescence image of host squid hemocytes incubated with inventive nanocomposites including one or more SPION and one or more QD having emission wavelength at 610 nm as detected by brightfield and fluorescence microscopy measured in the FITC channel.

FIG. 16A is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MultiDot peak emission Ab-CD”) including the emission of MD420-Ab-CD.

FIG. 16B is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD440-Ab-CD.

FIG. 16C is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD450-Ab-CD.

FIG. 16D is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD470-Ab-CD.

FIG. 16E is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD520-Ab-CD.

FIG. 16F is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD580-Ab-CD.

FIG. 16G is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD580-Ab-CD.

FIG. 16H is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD655-Ab-CD.

FIG. 16I is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD700-Ab-CD.

FIG. 17A is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD580-Ab-CD.

FIG. 17B is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD610-Ab-CD.

FIG. 17C is an flow cytometer emission plot of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission of MD655-Ab-CD.

FIG. 18A is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD420-Ab-CD.

FIG. 18B is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD450-Ab-CD.

FIG. 18C is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD480-Ab-CD.

FIG. 18D is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD580-Ab-CD.

FIG. 18E is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD610-Ab-CD.

FIG. 18F is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD655-Ab-CD.

FIG. 18G is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the histogram of MD700-Ab-CD.

FIG. 19A depicts flow cytometer emission profile of human lymphocytes CD4 labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots” abbreviated in the legend “MD peak emission Ab-CD”) including MD585-Ab-CD4.

FIG. 19B depicts flow cytometer emission profile of human lymphocytes CD4 bound to mouse anti-human CD4 antibody labeled with Starbright 570 dye.

FIG. 20A depicts a comparison of flow cytometer emission plots of polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab” abbreviated in the legend “MD peak emission Ab”) including the emission plots of MD440-CD19 (clone HIB19) ad MD520-CD3 (clone UCHT1) to flow cytometer emission plots of Fluorescein isothiocyanate (FITC) linked to CD8 antibody (clone UCHT4) and Brilliant Violet 421 linked to CD4 antibody (clone RPAT4).

FIG. 20B depicts a comparison of flow cytometer emission plots of polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab” abbreviated in the legend “MD peak emission Ab”) including the emission plots of MD580-CD8 (clone HIT8a), MD610-CD19 (clone HIB19) and MD640-CD45 (clone HI30) to flow cytometer emission plots of Allophycocyanin linked to CD3 antibody (clone OKT3) and Phycoerythrin linked to CD4 antibody (clone RPAT4).

FIG. 20C depicts a spillover spread matrix representing the spread of MD440-CD19 (clone HIB19), MD520-CD3 (clone UCHT1), MD580-CD8 (clone HIT8a), MD610-CD19 (clone HIB19), MD640-CD45 (clone HI30), Fluorescein isothiocyanate (FITC) linked to CD8 antibody (cline UCHT4), Brilliant Violet 421 linked to CD4 antibody (clone RPAT4), Allophycocyanin linked to CD3 antibody (clone OKT3) and Phycoerythrin linked to CD4 antibody (clone RPAT4).

FIG. 21A depicts the superimposition of flow cytometry emission plots of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells” abbreviated in the legend “MD peak emission Ab-CD”) including the emission plot of MD655-Ab-CD4 and the flow cytometry emission plots of human lymphocytes labeled with Brilliant Ultraviolet 661 linked to CD4 antibody and Allophycocyanin linked to CD4 antibody.

FIG. 21B depicts a spillover spread matrix representing the spread of human lymphocytes labeled with Brilliant Ultraviolet 661 linked to CD4 antibody, Brilliant Violet 421 linked to CD4 antibody, PerCP-Cy5.5 695 linked to CD4 antibody, and Allophycocyanin linked to CD4 antibody.

FIG. 21C depicts a spillover spread matrix representing the spread of human lymphocytes labeled with Brilliant Violet 421 linked to CD4 antibody, MD655-Ab-CD4, PerCP-Cy5.5 695 linked to CD4 antibody, and Allophycocyanin linked to CD4 antibody.

FIG. 22A is a flow cytometer histogram of human lymphocytes labeled with a polymer encapsulated nanoparticles encompassed by FIG. 1, Block 1B including one or more QD encapsulated in a polymer obtained by the polymer synthesis process shown in FIG. 2, Blocks 2A through 2I by use of electrohydrodynamic mixing mediated-nanoprecipitation as illustrated in FIG. 5 (“PENP MultiDots-Ab Cells (channel)” abbreviated in the legend “MD peak emission Ab-CD”) including the histogram MD420-Ab-CD19 (V1), MD440-Ab-CD3 (V20, MD470-Ab-CD4 (V4), MD520-Ab-CD8 (V6).

FIG. 22B depicts a spillover spread matrix representing the spread of MD420-Ab-CD19 (V1), MD440-Ab-CD3 (V20, MD470-Ab-CD4 (V4), MD520-Ab-CD8 (V6).

### V. DETAILED DESCRIPTION OF THE INVENTION

The present invention may be understood by reference to the following detailed description of aspects of the invention and the examples included therein and to the figures and their previous and following description. Compounds, compositions, articles, devices, or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments for the purpose of enabling a person of ordinary skill in the art to make and use a numerous and wide variety of embodiments of the invention, even if not explicitly disclosed. Unless defined otherwise, all technical and scientific terms used herein have the same meaning as commonly understood by one of ordinary skill in the art to which this invention belongs. Although any methods and materials similar or equivalent to those described herein can be used in the practice of the present invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein may be different from the actual publication dates, which may need to be independently confirmed.

As used in the specification and the appended claims, the singular forms “a,” “an” and “the” include plural referents unless the context clearly dictates otherwise. Thus, for example, reference to “a component,” “a polymer,” or “a particle” includes mixtures of two or more such components, polymers, or particles, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, as illustrative examples, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

Disclosed are the components to be used to prepare the compositions of the invention as well as the compositions themselves to be used within the methods disclosed herein. These and other materials are disclosed herein, and it is understood that when combinations, subsets, interactions, and groups of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, elements in methods of making and using the compositions of the invention. Thus, if there are a variety of additional elements that can be performed it is understood that each of these additional elements can be performed with any specific embodiment or combination of embodiments of the methods of the invention. It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Process Overview

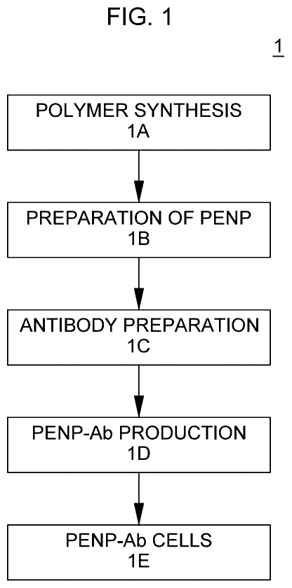

Now, with primary reference to FIG. 1, a block flow diagram provides a general overview of the nanomaterial polymer encapsulation system (1) including one or more of: polymer synthesis (Block 1A) of a nanoparticle (“NP”) (2) encapsulation polymer (“P”) (3); formation of polymer encapsulated nanoparticles (“PENP”) (4) (Block 1B); preparation of antibodies, half-antibodies, antibody fragments (individually or collectively “Ab”) (5) (Block 1C); preparation of PENP antibody conjugates (“PENP-Ab”) (6) (Block 1D); PENP-Ab cell labeling (PENP-Ab-Cells”) (7), flow cytometry analysis (8) and imaging of PENP-Ab-Cells (7) (Block 1E).

### Polymer Synthesis.

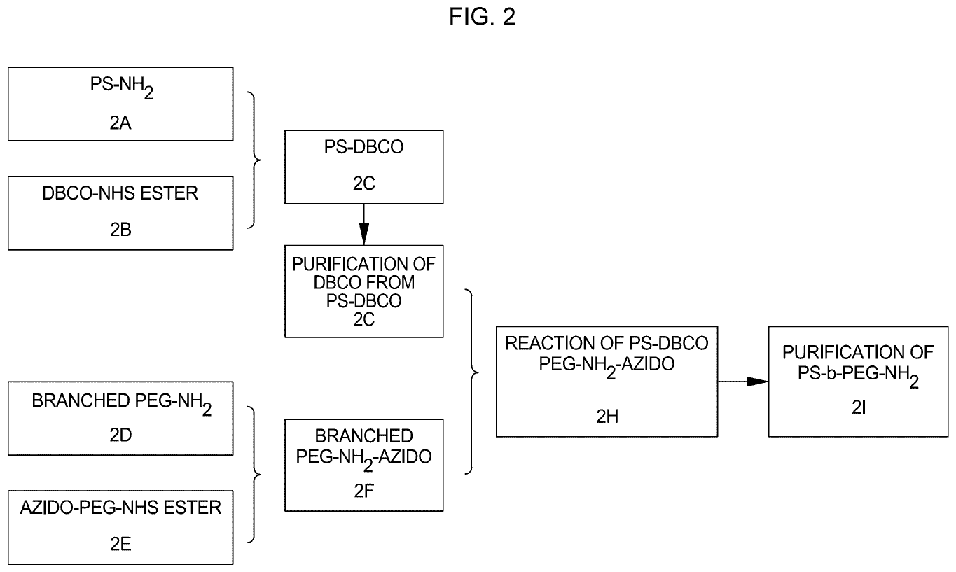

Now, with primary reference to FIG. 2, a block flow diagram provides an overview of an illustrative polymer synthesis process of FIG. 1, Block 1A. Referring to Block 2A of the process, aminopolystyrene (“PS—NH₂) having linear formula (C₈H₈)ₙCH₅N, wherein the PS molecular weight can occur in a range of about 1.5 kDa to about 40 kDa, can be obtained from Polymer Source, Inc., PN P3965-SNH₂, comprising Formula I. Particular embodiments can include PS-NH₂ having a number averaged molecular weight of about 9.5 kDa.

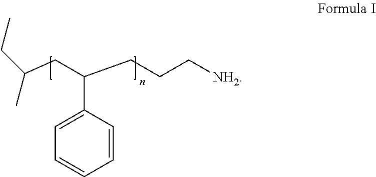

Now, referring to FIG. 2, Block 2B of the polymer synthesis process, dibenzocylooctyne-N-hydroxysuccinimidyl ester (“DBCO-NHS Ester”) having liner formula C₂₃H₁₈N₂O₅ (CAS No.: 1353016-71-3) and having a molecular weight of 402.40 g/mol can be obtained from Click Chemistry Tools, PN A133-100 comprising Formula II.

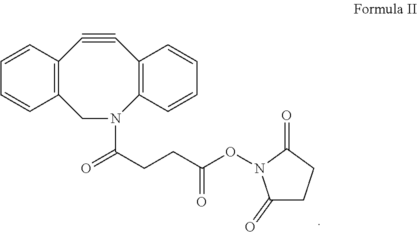 [SMILES: O=C(CCC(=O)N1Cc2ccccc2C#Cc2ccccc21)ON1C(=O)CCC1=O]

Now, referring to FIG. 2, Block 2C of the polymer synthesis process, PS—NH₂ can be reacted with DBCO-NHS Ester to produce polystyrene-dibenzocylooctyne (“PS-DBCO”) comprising Formula III.

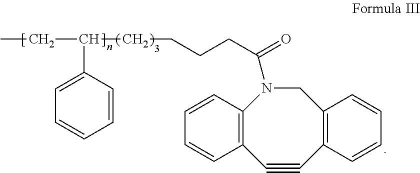

An example of a scalable procedure for the production of PS-DBCO can include thawing PS—NH₂ 20 mg for 15 minutes at room temperature (“RT”) and thawing DBCO-NHS-Ester 10 mg for 15 minutes (“min.”) at RT. Aliquot 1 mL of toluene (C₆H₅CH₃) (CAS No.: 108-88-3) to the PS—NH₂ 20 mg and vortex for 15 minutes at 500 RPM. Centrifuge DBCO at 1500 relative centrifugal force (“RCF”) (RCF=(RPM)²×1.118×10⁻⁵×r) for 30 seconds (“sec.”) at about 25° C. (about 77° F.). Aliquot 1 mL of toluene to the DBCO. Sonicate the DBCO for 5 min. in RT water. Transfer 1 mL of DBCO in toluene to PS—NH₂ in toluene. Vortex DBCO in PS—NH₂ mixture for 16 to 24 hours (“hr.”) at 500 RPM at RT.

Now, referring to FIG. 2, Block 2D of the polymer synthesis process, an 8-arm polyethylene glycol-amine (“PEG₈₋ₐᵣₘ-NH₂”) comprising a multi-arm PEG derivative with amine groups at each terminal of the eight arms connected to one hexaglycerol core having linear formula R(O(CH₂CH₂O)ₙCH₂CH₂NH₂)₈ and a number averaged molecular weight of about 19.5 kDa can be obtained from Nanosoft Polymers, PN 2443, comprising Formula IV and further shown in Formula V.

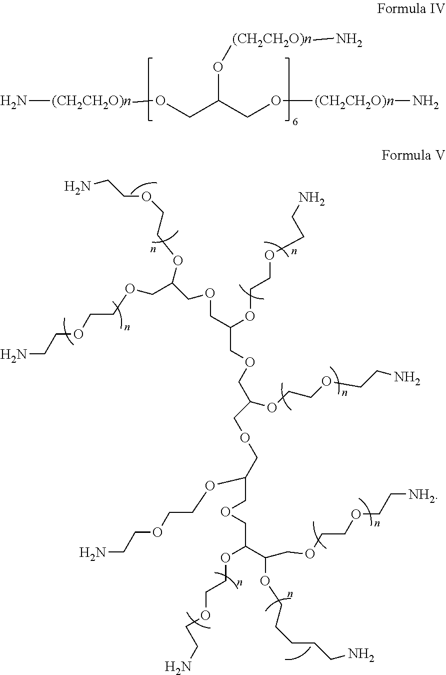

In particular embodiments, a 4-arm polyethylene glycol-amine, a 6-arm polyethylene glycol-amine, or an 8-armpolyethyleneglycol-amine, or combinations thereof, can also be utilized having corresponding PEG molecular weights of 10 kDa, 20 kDa, or 40 kDa.

Now, referring to FIG. 2, Block 2E of the polymer synthesis process, an azido-d-polyethylene glycol 4-N-hydroxysuccinimidyl ester (“Azido-PEG₄-NHS Ester”) having a molecular weight of 388.37 g/mol (CAS No.: 944251-24-5) can be obtained from Click Chemistry tools PN AZ103-100 comprising Formula VI.

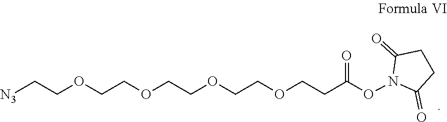 [SMILES: [N-]=[N+]=NCCOCCOCCOCCOCCC(=O)ON1C(=O)CCC1=O]

Now, referring to FIG. 2, Block 2F of the polymer synthesis process, PEG₈ₐᵣₘ-NH₂ can be reacted with Azido-PEG₄-NHS Ester to produce branched polyethylene glycol (“PEG-Amine-Azide”) comprising Formula VII.

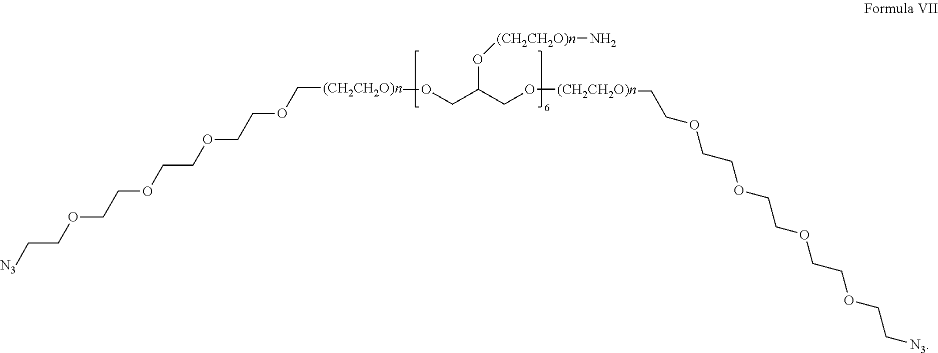

An example of a scalable protocol for the production of PEG-Amine-Azide can include thawing 40 mg of PEG₈₋ₐᵣₘ-NH₂ for 15 min. at RT and thawing 80 μl of Azido-PEG₄-NHS Ester (50 mM) for 15 min. at RT. Aliquot 400 μl of methanol (CAS NO.: 6756-1) to PEG₈₋ₐᵣₘ-NH₂ and vortex the PEG₈₋ₐᵣₘ-NH₂ for 15 min. at 500 RPM. Centrifuge Azido-PEG₄-NHS at 1500 RCF for 30 sec. at 25° C. (about 77° F.). Transfer the Azido-PEG₄-NHS 80 μl to the 400 μl of PEG₈₋ₐᵣₘ-NH₂. Vortex for 16 to 24 hr. at 500 RPM at RT.

Now, referring to FIG. 2, Block 2G of the polymer synthesis process, PS-DBCO obtained in Block 2C of the synthesis process can be purified by the following illustrative scalable procedure including transferring 400 μl of PS-DBCO to each of five centrifuge tubes. Aliquot 800 μl of methanol to each of the five centrifuge tubes. Mix PS-DBCO in methanol by inversion of each of the five centrifuge tubes. Centrifuge the PS-DBCO in methanol for 5 min. at 20,000 RCF at 15° C. to 25° C. (about 59° F. to about 77° F.). Decant the supernatant from each of five centrifuge tubes. Add 400 μl of toluene to each of the five centrifuge tubes. Place the five centrifuge tubes in a water bath at 37° C. (about 98.6° F.) for 2 min. Dissolve the PS-DBCO pellet in 400 μl toluene by mixing with a pipette. Add 800 μl of methanol to each of the five centrifuge tubes. Invert each centrifuge tube to mix contents. Centrifuge at 20,000 RCF for 5 min. at 15° to 25° C. (about 59° F. to about 77° F.). Decant the supernatant from each of five centrifuge tubes. Add 400 μl of toluene to each of the five centrifuge tubes. Place the five centrifuge tubes in a water bath at 37° C. (about 98.6° F.) for 2 min. Dissolve the PS-DBCO pellet in toluene by mixing with a pipette.

Now, referring to FIG. 2, Block 2H of the polymer synthesis process, PS-DBCO obtained in Block 2G of the synthesis process can be reacted with PEG-Amine-Azide obtained in Block 2F of the polymer synthesis process to produce polystyrene-b-poly(ethylene glycol) amine (“PS-b-PEG-NH₂”) comprising Formula VIII.

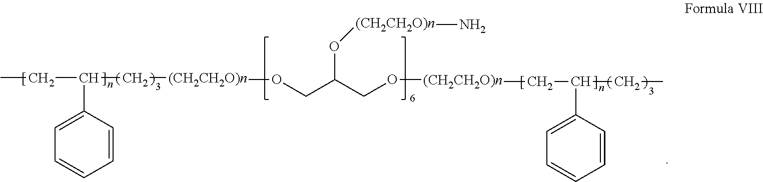

In the instant illustrative example of Block 2H, the PS-DBCO in toluene of all five centrifuge tubes from Block 2G and PEG-Amine-Azide in methanol obtained in Block 2F can be transferred to a 4 mL glass vial. Vortex the mixture of PS-DBCO and PEG-Amine-Azide at 500 RPM at RT for 16 to 24 hr. to produce PS-b-PEG-NH₂ comprising Formula VIII. The PS-b-PEG-NH₂ can comprise PS having molecular weights ranging from about 1.5 kDa to about 40 kDa and can comprise PEG having molecular weights ranging from about 10 kDa to about 40 kDa.

Referring to FIG. 2, Block 2I of the synthesis process, the PS-b-PEG-NH₂ obtained in Block 2H can be purified and dried by the following illustrative scalable procedure including cooling hexane (CAS No.: 110-54-3) at −20° C. (about −4° F.) for 15 min. Aliquot approximately 1.25 mL of PS-b-PEG-NH₂ obtained in Block 2I of the synthesis process into a 15 mL centrifuge tube. Slowly add 12 mL of hexane to the centrifuge tube containing PS-b-PEG-NH₂. Mix the PS-b-PEG-NH₂ in toluene/hexane by gently tilting the centrifuge tube 10 to 15 times. Place the centrifuge tube at −20° C. (about −4° F.) for 5 min. to cool the PS-b-PEG-NH₂ in toluene/hexane. Decant the supernatant from the PS-b-PEG-NH₂ pellet. Aliquot 1 mL of tetrahydrofuran to the centrifuge tube. Place the centrifuge tube in a water bath at the 37° C. (about 98.6° F.) for 2 min. Dissolve the PS-b-PEG-NH₂ pellet by mixing with a pipette. Slowly add 6 mL of cold hexane to the centrifuge tube. Gently tilt the centrifuge tube 10 to 15 times until PS-b-PEG-NH₂ precipitates as flakes are visualized with clear supernatant. Place the centrifuge tube at −20° C. (about −4° F.) for 5 min. Decant the supernatant from the PS-b-PEG-NH₂ pellet. Place the centrifuge tube containing the PS-b-PEG-NH₂ pellet at RT for 24 hr. to remove excess solvents and to obtain a dry PS-b-PEG-NH₂. Store at −20° C. (about −4° F.).

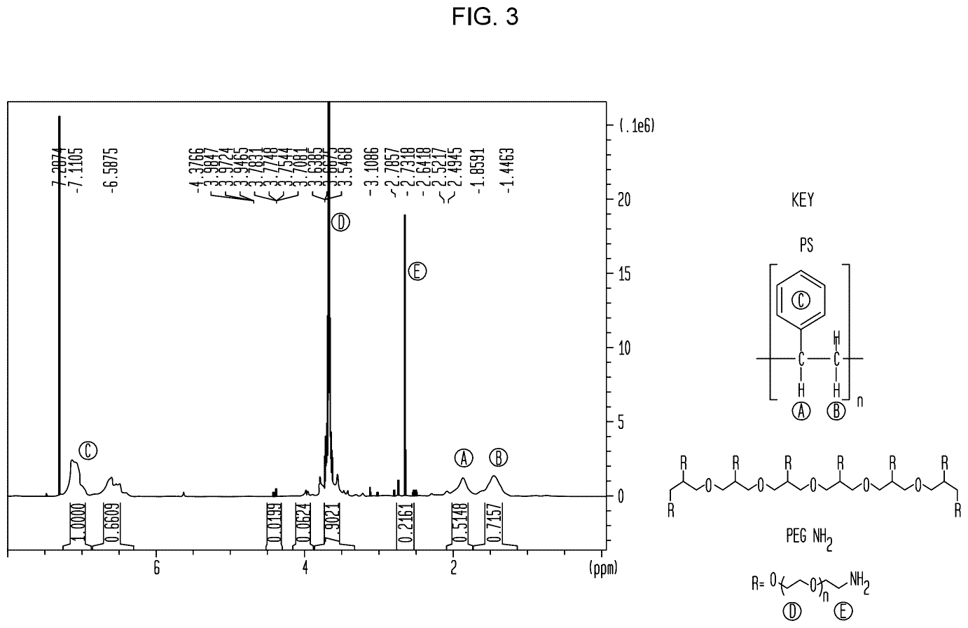

Now, referring primarily to FIG. 3, the structure of the PS-b-PEG-NH₂ obtained in Block 2I of the synthesis process can be analyzed using nuclear magnetic resonance (“NMR”). The NMR spectrum shown in FIG. 3 validates the molecular structure obtained by the polymer synthesis process shown in FIG. 2, Blocks 2A through 2I, and above disclosed polymer synthesis procedure, is purified PS-b-PEG-NH₂ comprising formula VIII.

The illustrative example of the polymer PS-b-PEG-NH₂ obtained in Block 2I is not intended to preclude embodiments of the PS-b-PEG including functional groups other than NH₂. Other functional groups can include as examples, one or more of: acrylate, maleimide, vinylsulfone, azide, biotin, carboxyl, thiol, alkyne, hydrazide, N-hydroxysuccinimide ester, and nitrophenyl carbonate, and combinations thereof, or embodiments including other similar or equivalent polymers including one or more functional groups.

### Formation of Polymer Encapsulated Nanoparticles.

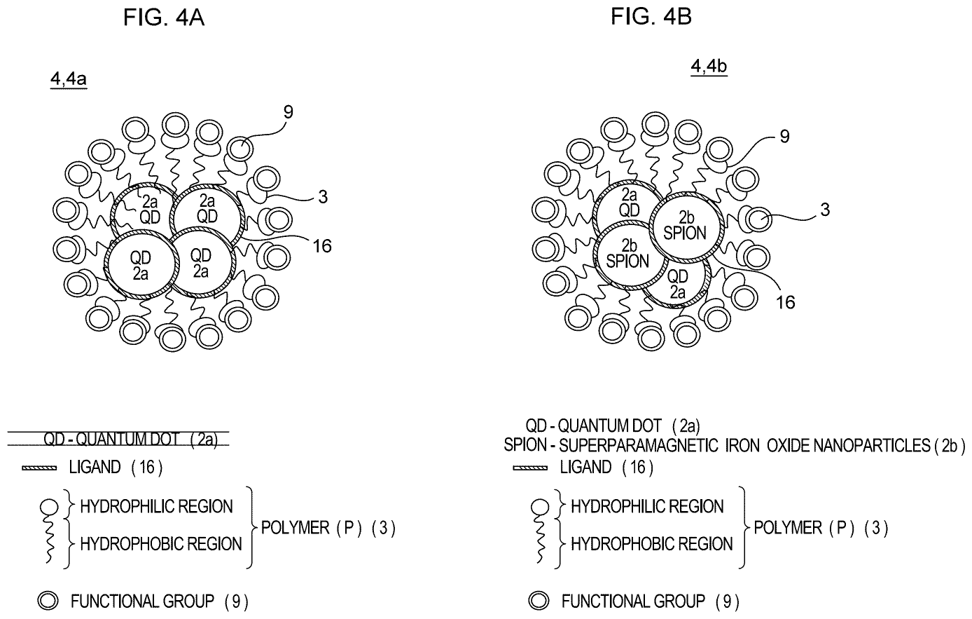

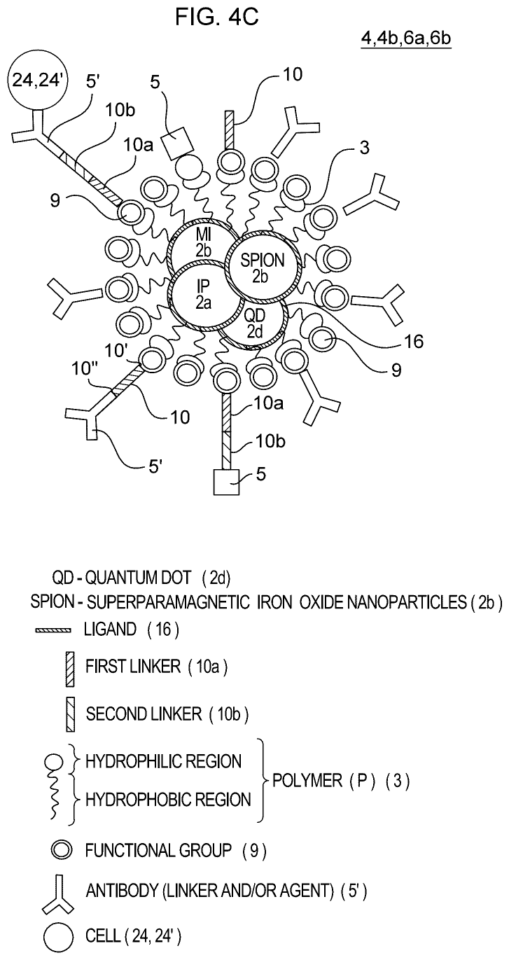

Now, with primary reference to FIG. 4A which illustrates a particular embodiment of PENP (4) including one or more QD (2a) encapsulated in a polymer (“P”) (3) having a hydrophobic region which can associate or coordinate with the one or more QD (2a) and a hydrophilic region including a functional group (9) which can associate with an aqueous environment, and in particular embodiments, the polymer (P) (3) can comprise PS-b-PEG-NH₂ obtained in Block 2I, which by solution based association can produce polymer encapsulated nanoparticles including one or more QD (2a) (“PENP-MultiDots”) (4a).

Now, with primary reference to FIG. 4B, which illustrates a particular embodiment of PENP (4) including one or more QD (2a) and one or more SPION (2b) encapsulated in a polymer (“P”) (3) having a hydrophobic region which can associate or coordinate with the one or more SPION (2b) and optionally one or more QD (2a) and a hydrophilic region including a functional group (9) which can associate with an aqueous environment, and in particular embodiments, the polymer (P) (3) can comprise PS-b-PEG-NH₂ obtained in Block 2I, which by solution based association can produce PENP (4) including one or more SPION (2b) and one or more QD (2a) (“PENP-MagDots”) (4b).

Now, with primary reference to FIG. 4C, a numerous and wide variety of agents (5) can be conjugated to PENP-MultiDots (4a) and/or PENP-MagDots (4b) using the functional group (9) of the polymer (3), and in particular embodiments, the amine afforded by PS-b-PEG-NH₂. The example of PENP-MagDots (4b) in FIG. 4C illustrates that one or more agents (5) can be conjugated to PENP-MultiDots (4a) and/or PENP-MagDots (4b) by activating the functional group (9), including as illustrative examples: one or more linkers (10, 10a, 10b), a polyethylene glycol, a fluorescent probe, an aptamer, a vitamin, a radioactive isotope, a contrast media, a surface charge modifier, a lectin, a protein, a peptide, a cell surface receptor, a cell coat, and combinations thereof. Specifically, in particular embodiments the functional group (9) can be utilized to directly or indirectly through one or more linkers (10, 10a, 10b) couple an antibody or antibody fragment (Ab) (5′) to produce PENP antibody conjugates (6) including as illustrative examples PENP-MultiDots-Ab (6a) and PENP-MultiDots-Ab (6b).

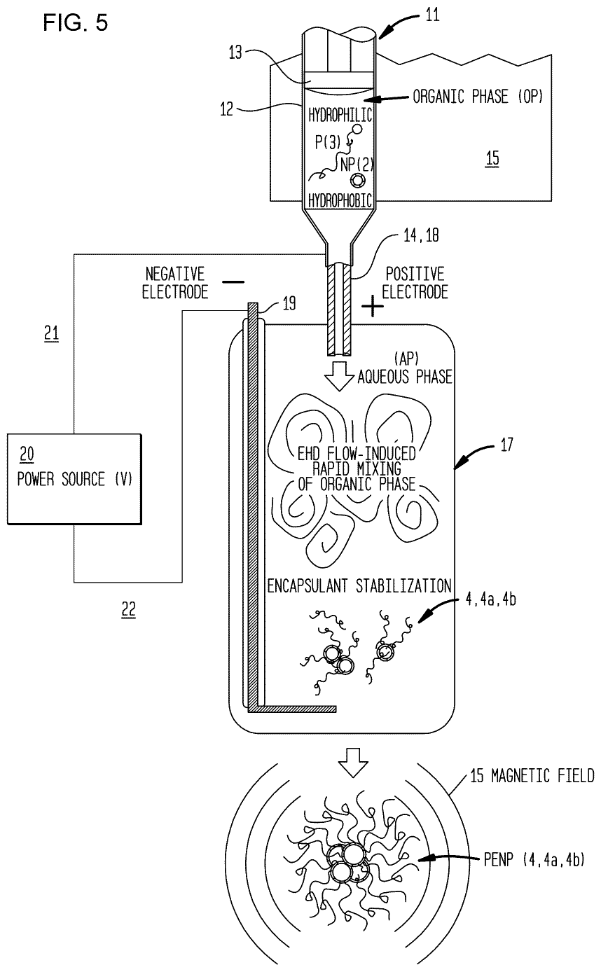

Now, with primary reference to FIG. 5, scalable, solution-based production of PENP (4) encompassed by the invention, including, but not necessarily limited to, PENP-MagDots (4b) or PENP-MultiDots (4a), can be prepared by conventional self-assembly, flash nanoprecipitation (FNP) comprising rapid turbulent mixing generated by high velocity flows, as described by Yanjie Zhang, a Aaron R. Clapp, RSC Advances, Issue 89, 2014 “Preparation of quantum dot-embedded polymeric nanoparticles using flash nanoprecipitation”, or by rapid mixing induced by electrohydrodynamics (“EHD”): EHD mixing mediated-nanoprecipitation (“EHD-NP”). Kil Ho Lee, Guolingzi Yang, Barbara E. Wyslouzil and Jessica O. Winter, ACS Appl. Polym. Mater. 2019, 1, 4, 691-700, each incorporated by reference herein.

The illustrative EHD mixing system of FIG. 5 can include a syringe (11) having a syringe barrel (12) fitted with a sliding syringe plunger (13), and a syringe needle (14). A syringe pump (15) configured drive the syringe plunger (13) to deliver an organic phase of water-miscible nonpolar aprotic solvents (“OP”) including solubilized (P) (3) and NP (4) whether QD (2a) and/or SPIONS (2b) (collectively the “inorganics”) at a predetermine volumetric flow rate into an aqueous phase (“AP”). The illustrative example of a syringe (11) and a syringe driver (15) is not intended to preclude other appliances useful in delivering the OP into the AP at a predetermined volumetric flow. The concentration of inorganics per unit volume of the OP can be about 0.1 volume to volume (“v/v”) to about 0.5 v/v. The concentration of P (3) to QD (2a) and/or SPION (2b) in the OP by mass can be about 1:1 to about 4:1. The amount of P (3) can be adjusted to obtain a PENP (4) having a substantially uniform hydrodynamic diameter (“HD”) that occurs in size range of about 40 nanometers (“nm”) to about 500 nm. In embodiments in which the QD (2a) and/or the SPIONS (2b) are passivated with a ligand (16), the ligand mass relative to the total inorganics mass can be about 20% to 40% by mass. A ligand mass percent greater than 40% can interfere with the assembly of the PENP (4).

A non-electrically conductive container (17) can hold the AP, typically distilled or deionized water. A positive electrode (18) and a negative electrode (19) can be introduced about 1 cm apart in the AP held by the non-electrically conductive container (17). In particular embodiments the syringe needle (14) can, if electrically conductive, act as the positive electrode (18). A voltage source (20) can supply a voltage (“V”) to the positive terminal (18) to generate an electrical field between the positive electrode (18) and negative electrode (19) in the AP. The syringe plunger (13) can be driven to introduce the OP into the AP at a consistent flow rate of about 8 mL h⁻¹ to about 15 mL h⁻¹. Voltage (V) can be adjusted between about −1 kilovolt (“kV”) and about −2.5 kV. The electric field can generate a fine dispersion of OP in the AP to produce PENP (4), PENP-MagDots (4b), or PENP-MultiDots (4a) of substantially uniform size. The resulting size of the PENP (4), PENP-MagDots (4b), or PENP-MultiDots (4a) can increase with increasing 5 V and/or concentration of inorganics per unit volume of the OP. The resulting PENP-MagDots (4b) can be subsequently isolated by influence of a magnetic field (15).

### Quantum Dots and Quantum Dot Sets.

QDs (2a) comprise inorganic semiconductor nanocrystals comprising an inorganic core semiconductor material (2a′) (also referred to as a “core material”) surrounded by a shell semiconductor material (2a″) (also referred to as a “shell material”) having a different band gap (annotated as “core material/shell material”) (as an example CdS/ZnS). QD (2a) can have a size that typically occurs in the range of 1 nm to 10 nm. QD (2a) can exhibit size-variable emission color due to the quantum confinement effect, where smaller QD (2a) emit at higher energy (lower wavelength) and larger QD (2a) emit at lower energy (higher wavelength) for a given composition. Accordingly, QD (2a) can absorb over a broad range and have photoluminescence emission over a narrow range which can be tuned depending on the material from which the QD (2a) is made and the size of the QD (2a). Combinations of various elements in II-VI, IV-VI, IIIV groups and perovskite type materials have been found to exhibit these properties on the nanoscale. By varying the size and composition of the QD (2a), the band-gap of the QDs (2a) can be tuned resulting in emission spanning the entire visible to infra-red spectrum. See for example: 6-Quantum Dot Materials, Devices and Their Applications in Photomedicine, Quantum Materials, Devices, and Applications Pages 155-200 (2023). Illustrative examples of QD (2a) core/shell compositions, and combinations thereof, suitable for use in embodiments of the PENP-MultiDots (4a) can include one or more of: CdS/ZnS, CdSSe/ZnS, CdSe/ZnS, CdTe/ZnS, and CdSeTe/ZnS each having an emission photoluminescence occurring in the range of 420 nm to 880 nm; CuInZnS/ZnS, 540 to 660 nm; and PbS/CdS, 700 nm to 900 nm. However, these examples are not intended to preclude embodiments having other QD (2a) core/shell compositions.

Purified QD (2a) can be bare or can be capped to control QD particle size and/or to prevent QD agglomeration. QD (2a) synthesized by prototypical hot-injection method can be capped with a ligand (16), such as, oleylamine and oleic acid after QD purification. ¹H NMR spectroscopy analysis evidence that ligand binding can be highly dynamic, and that oleylamine selectively binds to the surface as oleylammonium bromide in an NC(X)₂ binding motif. Only in the presence of excess oleylamine added after purification does oleic acid bind to the surface, in the form of oleylammonium oleate. Protesescu L, Yakunin S, Bodnarchuk M I, Krieg F, Caputo R, Hendon C H, Yang R X, Walsh A, Kovalenko M V, Nano Lett. 2015, 15, 3692-3696, hereby incorporated by reference herein. While the use of oleylamine and oleic acid as a capping ligand (16) is suitable for embodiments of the invention, this is not intended to preclude embodiments using other capping ligand(s) (16), as examples, trioctylphosphine oxide, L-histidine, chitosan, polyvinyl alcohol, polyvinylpyrrolidone or combinations thereof. QDs (2a) within QD sets in accordance with the invention can be capped with organic ligands, as illustrative examples, oleic acid, oleylamine or thiol compounds making them soluble in organic solvents such as toluene.

Typically, bare QD (2a) or capped QD (2a) reach a required level of water solubility and biocompatibility by surrounding the QD (2a) with a P (3) to produce PENP (4). A P (3) suitable for use with embodiments of the invention can include various embodiments of PS-b-PEG-NH₂ obtained in Block 2I of the synthesis process, above described.

Embodiments of PENP (4) can be produced through the use of various combinations of one or more of: QD (2a), capping ligand (16), and PS-b-PEG-NH₂ having PS molecular weights ranging from about 1.5 kDa to about 40 kDa and PEG having molecular weights ranging from about 10 kDa to about 40 kDa.

It has been discovered that embodiments of QDs (2a) in accordance with the invention can comprise Cadmium Selenide (“CdSe”) a II-VI semiconductor material formed from Group II (Cadmium) and Group VI (Selenide) elements that emit in visible wavelength region ranging from 450 nm to 650 nm at room temperature. The intrinsic bandgap of CdSe is about 1.70 eV to about 1.74 eV emitting higher energy and shorter wavelengths in the visible spectrum. Cadmium Tellurium Selenide (“CdTeSe”), a II-VI semiconductor material, has an intrinsic bandgap of about 1.44 eV to about 1.56 eV resulting in emission in the short wavelength near IR range at about 700 nm at room temperature. CdSe and CdTeSe cores can include an epitaxial shell ZnS/ZnSe which protects and stabilizes the unique optical properties of the CdSe/CdTeSe core from potentially damaging environments, while reducing surface trap states and improving the photoluminescence yields to >70%. Depending on the emission wavelength, the core/shell structures typically have a physical diameter of about 5 nm to about 12 nm with a polydispersity of +/−0.5 nm. The emission peaks of the group II quantum dots (2a) measured at full width at half maximum can be separated by <25 nm resulting in narrow emission peaks.

In particular embodiments, QDs (2a) in accordance with embodiments of the invention can be manufactured having a narrow spectral emission with average full width at half maximum emission peaks separated by about 20 nm in the visible spectrum of about 380 nm to about 700 nm. As an illustrative example, QDs (2a) each emitting one of five specific pure colors can be stacked within each 100 nm wavelength along the entire visible spectrum up to about 700 nm wavelength.

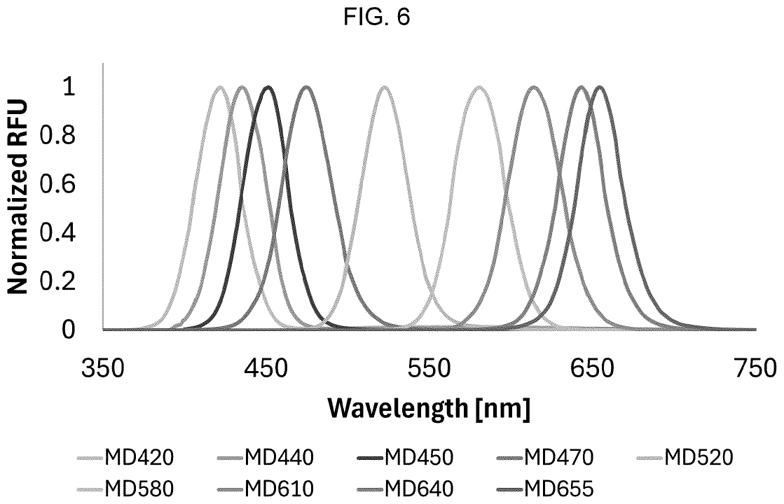

As shown in the illustrative example of FIG. 6, collectively, the properties of the QDs (2a) enable CdSe quantum dots emitting in the 450 nm to 650 nm range, when encapsulated as PENP (4), PENP-MagDots (4b), or PENP-MultiDots (4a), to be stacked at 20 nm intervals with minimal spectral bleed between adjacent emission channels separated by as little as 20 nm.

Additionally, it has been discovered that, Indium Arsenide (InAs) a III-V semiconductor material formed from Group III (Indium) and Group V (Arsenic) elements emit in the near infrared (“IR”) region from 800 nm to 1000 nm at room temperature. The intrinsic bandgap of InAs is about 0.35 eV to about 0.36 eV resulting in emitting lower energy and longer wavelength region in the infra-red region (“IR”). InAs can include epitaxial shell layers of InP/ZnS/ZnSe that result in improved photoluminescence quantum yields of >70%. Depending on the emission wavelength, the core/shell structures can have a physical diameter of about 3 nm to about 12 nm with the polydispersity of +/−0.5 nm. These near IR quantum dots (2a) have full width of half maxima of <120 nm resulting in broader emission peaks than the group II semiconductor quantum dots. In particular embodiments, QDs (2a) can be manufactured having a narrow spectral emission with emission peaks separated by about 50 nm in the visible spectrum of about 700 nm and 1000 nm

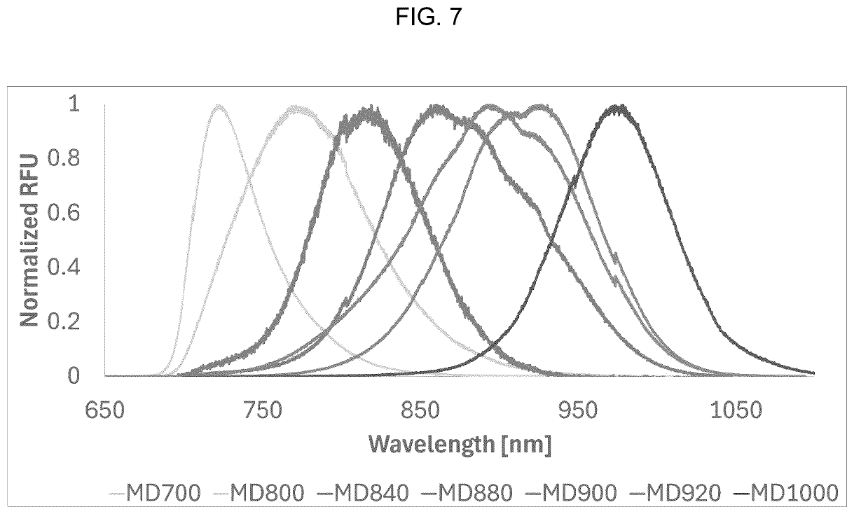

As shown in the illustrative example of FIG. 7, collectively, these properties enable InAs and/or CdTeSe quantum dots (2a) emitting in the 700-1000 nm range, when encapsulated as PENP (4), PENP-MagDots (4b), or PENP-MultiDots (4a), to be stacked with minimal spectral bleed between adjacent emission channels separated by as little as 50 nm.

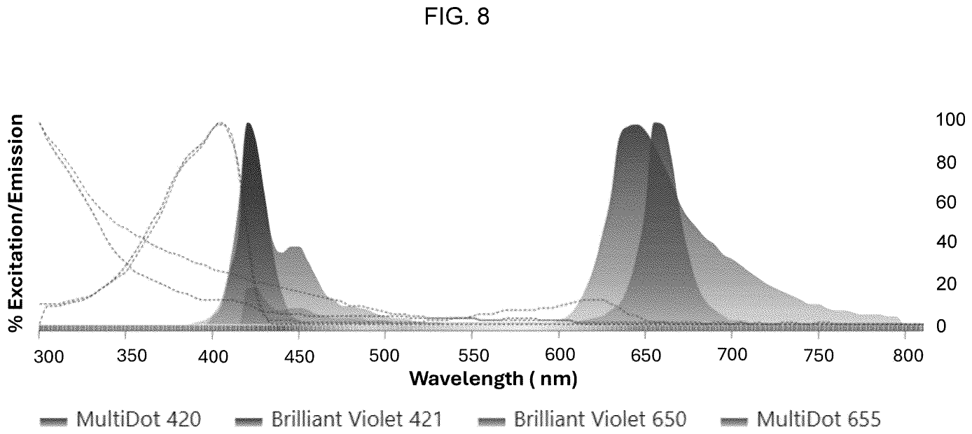

Now, with primary reference to FIG. 8, conventional reagents that have excitation emissions in visible spectrum have broad emission shoulders. As shown in the illustrative example, brilliant violet dyes, including Brilliant Violet 421 (BV421) can be excited by a 405 nm violet laser and emits at 421 nm and Brilliant Violet 650 (BV650) can be excited by a 405 nm violet laser and emits at 645 nm. However, the emission spectrum graph of Brilliant Violet 421 (BV421) and Brilliant Violet 650 include broad descending emission tails. By comparison, a QDs (2a) in accordance with the invention excited with a 405 nm violets laser emits at 420 nm and 655 nm respectively and have by comparison a substantially narrower emission peaks.

### PENP Multidots.

An illustrative example of a scalable method of production PENP (4), including but not necessarily limited to, PENP-MultiDots (4a), by EHD can include one or more of: constitute PS-b-PEG-NH₂ obtained in FIG. 2, Block 2I at 10 mg/mL into a first 1.5 mL tube. Introduce 240 μl of the desired QD at 5 mg/mL (2a) into a second 1.5 mL tube. Transfer 480 μl of acetone/methanol (60/40) to the QD (2a) in the second 1.5 mL tube. Centrifuge the second 1.5 mL tube containing the QD (2a) at 7000 RCF for 1 min, and then remove supernatant with a 200 μL pipette. Transfer 480 μL of anhydrous tetrahydrofuran (“THF”) to the second 1.5 mL tube containing the QD and mix thoroughly with a pipette. Introduce into a fresh 1.5 mL centrifuge tube 240 μl of THE, 240 μl QD in THE, and 120 μl of the solubilized PS-b-PEG-NH₂ to produce the OP for EHD.

Particular embodiments, EHD can be performed by cleaning the EHD mixing system syringe (11) three times with THF. Load about 0.6 mL of the OP into the syringe barrel (12). Mix the inorganics in the OP thoroughly while loading the syringe (11). Attach the syringe (11) to the syringe pump (15) and set the syringe pump (15) to generate a flow rate of the OP containing the inorganics from the syringe needle (14) in the range of about 11.00 mL/hr. to about 14.00 mL/hr. In particular embodiments the flow rate can be about 12.5 mL/hr. In particular embodiments the flow rate can be about 12.5 mL/hr. Prime the syringe needle (14) until a drop of the OP forms at the end of the syringe needle (14). Twice rinse a 20 mL glass vial (17) with distilled or deionized water (individually or collectively “DI water”). Introduce about 10 mL of DI water into the 20 mL glass vial. Submerge the syringe needle (14) into the 20 mL glass vial (17). Clean the negative electrode (19) by submerging in THE, wipe, and rinse with DI water. Place the negative electrode (19) into the AP contained in the 20 mL glass vial. Place the positive electrode (18) into the AP contained in the 20 mL glass vial (17). In particular embodiments the syringe needle (14), if electrically conductive, can act as the positive electrode (18). Observe that the positive electrode (18), the negative electrode (19), and the syringe needle (14) do not contact. Connect the positive lead (21) from the voltage source (20) to the positive electrode (18) or syringe needle (14) and connect the negative lead (22) from the voltage source (20) to the negative electrode (19). Verify that the voltage source (20) delivers about −1500 V.

The EHD-NP (4) produced by mixing the OP with the AP under influence of the electrical field can be concentrated using centrifugal filtration. The contents of the 20 mL glass vial (17) can be transferred to a 100 kDa cutoff centrifugal ultrafiltration column (“CUC”), as an example, SigmaAldrich PN UFC9010D Amicon® Ultra-15 Centrifugal Filter Unit. Centrifuge the CUC at 3000 RCF for 30 min. at about 25° C. (about 77° F.). Transfer 10 mL 50 mM sodium borate, 100 mM sodium phosphate, 7.3-7.5 pH (“borate buffer”) to the CUC. Centrifuge the CUC at 3000 RCF for 30 min. at about 25° C. (about 77° F.). Transfer EHD-NP filtrate from the CUC into a 1.5 mL microcentrifuge tube. Measure and record the volume of the collected EHD-NP filtrate. Transfer 15 μl of EHD-NP filtrate from the 1.5 mL microcentrifuge tube to a 1.5 mL tube and add 285 μl borate buffer. Transfer 290 μl of EHD-NP filtrate to a spectrophotometer cuvette. Measure and record optical density with a spectrophotometer at 450 nm (OD₄₅₀).

The EHD-NP filtrate may contain aggregates of PENP-MultiDots (4a) or aggregates of polymer (P) (3) lacking a QD (2a) (individually and collectively “aggregate”). The aggregate can be substantially removed from the EHD-NP filtrate to produce substantially pure PENP-MultiDots (4a). A scalable method for purification of PENP-MultiDots (4a) can include one or more of: transfer of 120 μl aliquots of the EHD-NP filtrate from the centrifugal ultrafiltration column to corresponding 1.5 mL tubes. Centrifuge the 1.5 mL tubes containing the EHD-NP filtrate at 3,000 RCF for 10 min. to pellet aggregates. Without disturbing the aggregate pellet, remove PENP-MultiDots (4a) containing supernatant. Measure and record the volume of the PENP-MultiDots supernatant. Mix 15 μl of PENP-MultiDots supernatant and 285 μl of borate buffer in 1.5 mL tube. Transfer 290 μl of the mixture to a spectrophotometer cuvette. Measure and record optical density with spectrophotometer at 450 nm (OD₄₅₀).

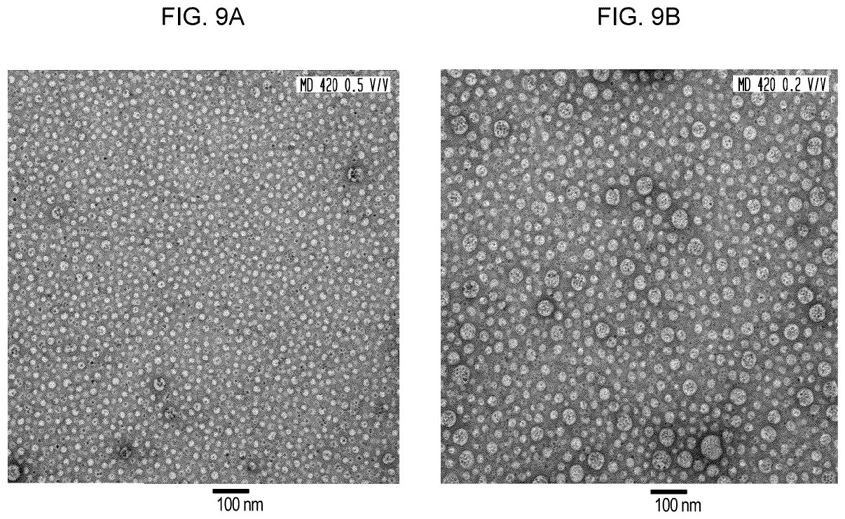

Now, with primary reference to FIG. 9A and FIG. 9B, the morphology of PENP-MultiDots (4a) produced by the above EHD method at concentration of P (3) and QD (2a) in the OP at 0.5 v/v and 0.2 v/v can be characterized by the use of transmission electron microscopy (“TEM”) to produce micrographs of the PENP-MultiDots (4a). PENP produced at concentration of QD (2a) in the OP at 0.5 v/v as shown in the example of FIG. 9A can load fewer QD (2a) per PENP (4) in comparison with PENP (4) produced at concentration of QD (2a) in the OP at 0.2 v/v as shown in the example of FIG. 6B that load more QD (2a) per PENP (4) which evidences that concentration of P (3) and QD (2a) in the OP can be adjusted to affect size and fluorescence per PENP-MultiDot (4a).

Embodiments of PENP-MultiDots (4a) evidence hydrodynamic diameter (“HD”) and polydispersity index (“PDI”) and size stability suitable for effective clinical and non-clinical applications. Hydrodynamic diameter and PDI can be obtained by performing dynamic light scattering measurements using NanoBrook 90 Plus particle size analyzer. Size histograms can be plotted using SigmaPlot (Systat Software Inc., San Jose, CA, U.S.A.), and size distributions can be fit to log-normal distributions.

HD is defined as the size of a hypothetical hard sphere that diffuses in the same fashion as that of the particle being measured. Though in practice, macromolecules or particles in solution are solvated, dynamic and non-spherical. Due to this, the diameter calculated from the particle's diffusional properties will signify the apparent size of the solvated/dynamic hydrated particle. The HD of the PENP-MultiDots (4A) produced by the above EHD method can vary depending on the parameters used during EHD to produce PENP (4). The TEM micrographs shown in FIG. 6A and FIG. 6B evidence PENP-MultiDots (4a) having substantially consistent HD of about 40 nm to about 50 nm; however, by varying the parameters used during EHD, PENP-MultiDots (4a) HD can vary in a range of about 40 nm to about 100 nm.

PDI is a representation of the distribution of size populations within a particle sample. The numerical value of PDI ranges from 0.0 for a perfectly uniform population within a particle sample to 1.0 for a highly polydisperse population within a particle sample. PDI values of 0.2 and below are deemed acceptable in practice for polymer-based nanoparticle materials. The calculations used for the determination of PDI are defined in the ISO standard documents 13321:1996 E and ISO 22412:2008. PENP-MultiDots (4a) can have a substantially consistent PDI of about 0.1 to about 0.2.

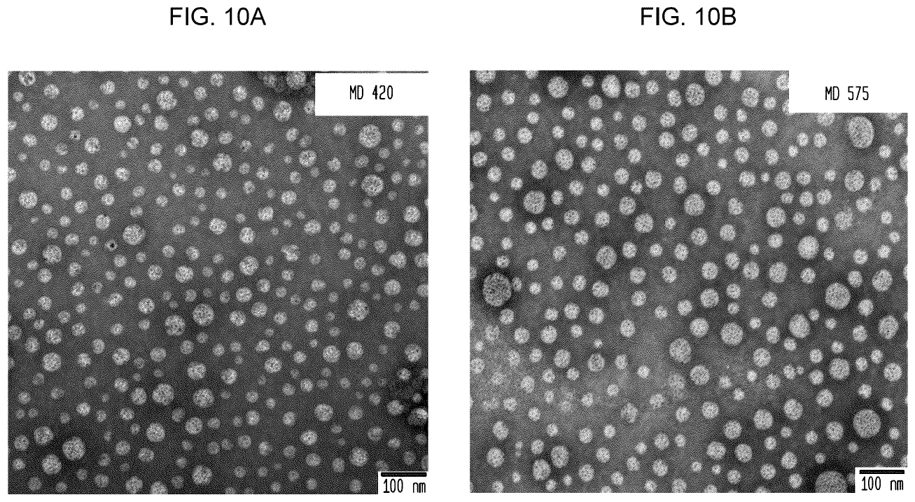

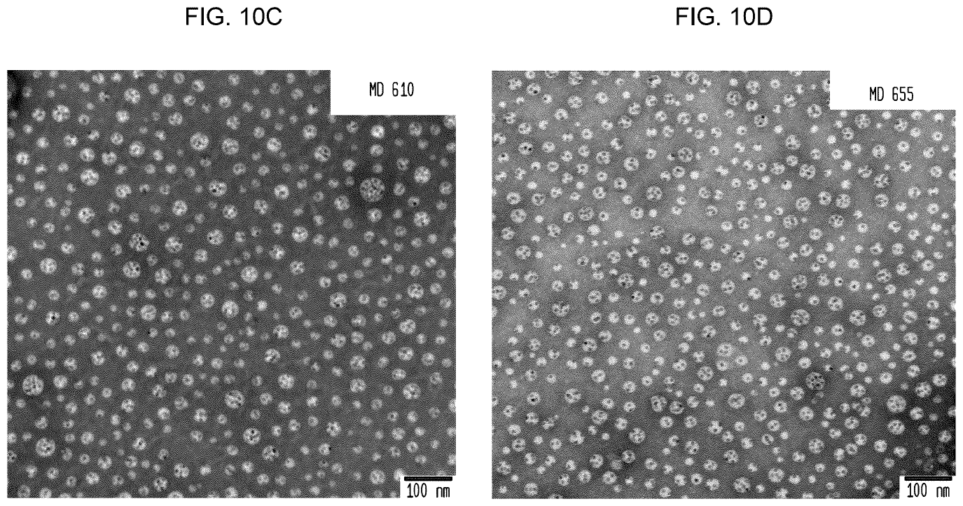

Now, with primary reference to FIGS. 10A through 10D, TEM micrographs evidence that PENP-MultiDots (4a) can be loaded with QD (2a) having a range of emission wavelengths in the visible and near infrared spectrum while in the range retaining a substantially uniform and narrow size distribution. In particular embodiments, the PENP-MultiDots (4a) can be loaded with QD (2a) having emission wavelengths occurring in the range of about 420 nm to about 880 nm or upconverting nanoparticles. The illustrative examples of FIGS. 10D through 10D, evidence PENP-MultiDots (4a) having an emission wavelength of 420 nm (as shown the example of FIG. 10A), an emission wavelength of 575 nm (as shown the example of FIG. 10B), an emission wavelength of 610 nm (as shown by the example of FIG. 10C), and an emission wavelength of 655 nm (as shown by the example of FIG. 10D). A long felt but unresolved concern with nanocomposites is the lack of uniform and narrow size distribution between nanocomposites including QD (2a) having different emission wavelengths. As an illustrative example, QD (2a) that have an emission wavelength at 420 nm can be smaller in size than QD (2a) that have an emission wavelength at 610 nm. Additionally, the larger the QD (2a) the brighter the QD (2a) for a given composition. Moreover, under substantially similar methods of preparation, nanocomposites load a greater number of QD (2a) that have an emission wavelength at 420 nm as compared to QD (2a) that have an emission wavelength at 610 nm. These differences between QD (2a) of varying emission wavelength in regard to one or more of: size, brightness and loading affinity under similar methods of preparation can result in nanocomposites that correspondingly differ in size and brightness. Embodiments of the nanocomposites including QD (2a) having different emission wavelengths can be produced by the inventive method in a uniform and narrow size distribution by adjusting the QD inorganic metal mass to polymer mass ratio in the organic phase (OP) introduced into the aqueous phase (AP) during EHD mixing. In embodiments of the PENP-MultiDots (4a), the QD inorganic metal mass to polymer mass can be adjusted between 1:1 to 1:2 to produce PENP-MultiDots (4a) having uniform and narrow size distribution between nanocomposites containing QD (2a) having different emission wavelengths in the range of 40 nm to 500 nm and having substantially similar brightness. In embodiments of the PENP-MagDots (4b), the QD and SPION inorganic metal mass to polymer mass can be adjusted between 1:2 to 1:4 to produce PENP-MagDots (4b) having uniform and narrow size distribution between nanocomposites containing QD having different emission wavelengths in the range of 40 nm to 500 nm and having substantially similar brightness. It is not intended that these illustrative examples preclude other embodiments of PENP-MultiDots (4a) which can be produced, as above described, having other mono-wavelength emission, dual-wavelength emission, or more than dual-wavelength emission depending on the one or more QD (2a) encapsulated in the polymer (P) (3).

### PENP-MagDots.

Superparamagnetic iron oxide nanoparticles (“SPION”) (2b) are small synthetic particles of maghemite having an empirical formula of Fe₂O₃, γ-Fe₂O₃ and a molar mass 159.69 g/mol (CAS No. 1309-37-1) or magnetite having an empirical formula of Fe₃O₄ and a molar mass of 231.53 g/mol (CAS NO. 1317-61-9), or combinations thereof, with a core ranging from about 5 nm to about 30 nm in diameter. In addition, mixed oxides of iron with transition metal ions such as copper, cobalt, nickel, and manganese, are known to exhibit superparamagnetic properties and also fall into the category of SPION (2b). However, magnetite and maghemite nanoparticles are the most widely used SPION (2b) in various biomedical applications. SPIONS (2b) exhibit the phenomenon of “superparamagnetism.” The particles that exhibit superparamagnetism, on application of an external magnetic field (15) (as shown in the example of FIG. 5), become magnetized up to their saturation magnetization, and on removal of the magnetic field, no longer exhibit any residual magnetic interaction. This property is size-dependent and generally arises when the size of nanoparticles reaches about 5 nm. At such a small size, these nanoparticles do not exhibit multiple domains as found in large magnets; on the other hand, they become a single magnetic domain and act as a “single super spin” that exhibits high magnetic susceptibility. Thus, on application of a magnetic field, these nanoparticles provide a stronger and more rapid magnetic response compared with bulk magnets with negligible remanence (residual magnetization) and coercivity (the field required to bring the magnetism to zero). Once the applied magnetic field is removed, the magnetic particles retain no residual magnetism at room temperature and are easily dispersed. Uncoated iron oxide NPs have very low solubility that can lead to precipitation due to gravitation forces and also a high rate of agglomeration under physiological conditions. Thus, to be used effectively, SPION can be capped with an amphiphilic coating.

In particular embodiments, SPION (2b) capped with a ligand (16), such as oleic acid, can have particle sizes in the range of about 5 nm to about 30 nm. Various SPION (2b) can be obtained from Ocean NanoTech, PN SOR05 to SOR30. Illustrative examples of embodiments, include SPION (2b) capped with oleic acid having particle sizes of 5 nm, 10 nm, 15 nm, and 20 nm. However, these illustrative SPION (2b) particle sizes are not intended to preclude the use of SPION (2b) having lesser or greater particle size, or combinations of SPION (2b) particle sizes, which can be used alone or in combination with one or more QD (2a) in embodiments of the PENP (4).

Typically, capped SPION (2b) in accordance with the invention reach a required level of water solubility and biocompatibility by surrounding the SPION (2b), or SPION/QD (2a, 2b) combination(s), with a polymer (3). A polymer (3) encapsulation layer suitable for use with embodiments of the invention include PS-b-PEG-NH₂ obtained in Block 2I of the synthesis process, above described. Embodiments of the invention can be produced through the use of various combinations of one or more of: SPION (2b) or SPION/QD (2a, 2b), capping ligand (16), and PS-b-PEG-NH₂ having PS molecular weights ranging from about 1.5 kDa to about 40 kDa and PEG having molecular weights ranging from about 10 kDa to about 40 kDa.

An illustrative example of a scalable method of production PENP-MagDots (4b) by EHD can include one or more of: PS-b-PEG-NH₂ obtained in FIG. 2, Block 2I at 10 mg/mL into a first 1.5 mL tube. Introduce 13 μl of the desired SPION (2b) particle from 25 mg/mL in toluene (e.g. 20 nm) into a second 1.5 mL tube. Transfer 52 μl of acetone/methanol (60/40) to the SPION in the second 1.5 mL tube. Mix by shaking. Centrifuge the second 1.5 mL tube containing the mixed SPION (2b) at 7000 RPM for 1 min. at about 25° C. (about 77° F.). Immediately remove supernatant with a 200 μl pipette. Immediately, transfer 13 μl of anhydrous tetrahydrofuran (“THF”) to the second 1.5 mL tube containing the SPION (2b) and mix thoroughly with a pipette. Transfer 65 μl of a QD (2a) (from 5 mg/mL in toluene) into third 1.5 mL micro centrifuge tube. Transfer 130 μl of acetone/methanol (60/40 v/v) to the third 1.5 mL microcentrifuge tube containing the QD. Tilt to gently mix the QD (2a). Centrifuge the third 1.5 mL microcentrifuge tube containing QD at 7000 R.P.M. for 1 minute at 25° C. (about 77° F.). Remove the supernatant with 200 μl pipette. Transfer 65 μl of THF to the third 1.5 mL microcentrifuge tube. Mix thoroughly with an air displacement pipette. To a 4 mL vial, add 322 μL of THF. Transfer 13 μL of the SPION (2b) in THF contained in the second 1.5 μL microcentrifuge tube into the 4 mL vial. Optionally, transfer 65 μL of the QD (2a) in THF contained in the third 1.5 mL microcentrifuge tube into the 4 mL vial. Transfer 100 μL of the PS-b-PEG-NH₂ into the 4 mL vial to produce the OP for EHD.

Particular embodiments, EHD can be performed by cleaning the EHD mixing system syringe (11) three times with THF. Load about 0.5 mL of the OP into the syringe barrel (12). Mix the inorganics in the OP thoroughly while loading the syringe (11). Attach the syringe (11) to the syringe pump (15) and set the syringe pump (15) to generate a flow rate of the OP containing the inorganics from the syringe needle (14) in the range of about 11.00 mL/hr. to about 14.00 mL/hr. In particular embodiment the flow rate can be about 12.5 mL/hr. Prime the syringe needle (14) until a drop of the OP forms at the end of the syringe needle (14). Twice rinse a 20 mL glass vial (17) with distilled or deionized water (individually or collectively “DI water”). Introduce about 10 mL of DI water into the 20 mL glass vial (17). Submerge the syringe needle (14) into the 20 mL glass vial (17). Clean the negative electrode (19) by submerging in THE, wipe, and rinse with DI water. Place the negative electrode (19) into the AP contained in the 20 mL glass vial (17). Place the positive electrode (18) into the AP contained in the 20 mL glass vial (17). In particular embodiments the syringe needle (14), if electrically conductive, can act as the positive electrode (18). Observe that the positive electrode (18), the negative electrode (19), and the syringe needle (14) do not contact. Connect the positive lead (21) from the voltage source (20) to the positive electrode (18) or syringe needle (14) and connect the negative lead (22) from the voltage source (20) to the negative electrode (19). Verify that the power source delivers about −1000 V.

The EHD-PENP (4) produced by mixing the organic phase mixed with the DI water under influence of the electrical field can be concentrated using centrifugal filtration. The contents of the 20 mL glass vial (17) can be transferred to a 100 kDa cutoff centrifugal ultrafiltration column available from SigmaAldrich, PN UFC9010D Amicon® Ultra-15 Centrifugal Filter Unit. Centrifuge at 2000 RCF for 30 min. at about 25° C. (about 77° F.). Transfer EHD-PENP (4) from the centrifugal ultrafiltration column to a 1.5 mL microcentrifuge tube. Measure and record the EHD-NP volume. Transfer 5 μL of EHD-PENP (4) filtrate from the 1.5 mL microcentrifuge tube to a 1.5 mL tube and add 295 μl borate buffer. Transfer 290 μl of EHD-PENP (4) to a spectrophotometer cuvette. Measure and record optical density with spectrophotometer at 450 nm (OD₄₅₀).

The EHD-PENP filtrate may contain aggregates of PENP (4) or aggregates of PENP lacking a SPION (Sb) or a QD (2a) (collectively “aggregate”). The aggregates can be substantially removed from the EHD-NP filtrate to produce substantially pure PENP-MagDots (4b). As one example, a scalable size exclusion process to purify PENP-MagDots (4b) can include: place a magnetic particle separation column (“MPSC”) in a magnetic field gradient (e.g. up to about 100-200 T/m). Transfer 300 μl of borate buffer into MPSC. Allow the borate buffer to pass through the MPSC. Introduce the EHD-NP filtrate into the MPSC in the magnetic field (23). Allow the filtrate liquid to pass through the MPSC. Transfer 300 μl of borate buffer into MPSC. Mix the EHD-NP in the borate buffer in the MPSC. Allow all of the borate buffer to pass through the MPSC. Extract the MPSC from the magnetic field gradient. Once the MPSC is extracted from the magnetic field gradient (23) the MPSC can no longer retain the PENP-MagDots (4b) which can then be eluted. Remove any liquid from MPSC tip. Place the MPSC in a PENP-MagDots (4b) collection tube labeled PENP-MagDots-Wavelength. Transfer 200 μl of borate buffer into the MPSC. Pipette to mix PENP-MagDots (4b) in the borate buffer in the MPSC. Collect PENP-MagDots (4b) into the PENP-MagDots-Wavelength collection tube. Mix collected PENP-MagDots (4b) thoroughly with an air displacement pipette. Measure and record the volume of the collected PENP-MagDots (4b). Combine 5 μl of PENP-MagDots (4b) and 295 μl of borate buffer. Transfer 290 μl of sample to spectrophotometer cuvette. Measure and record optical density with spectrophotometer at 450 nm (OD₄₅₀).

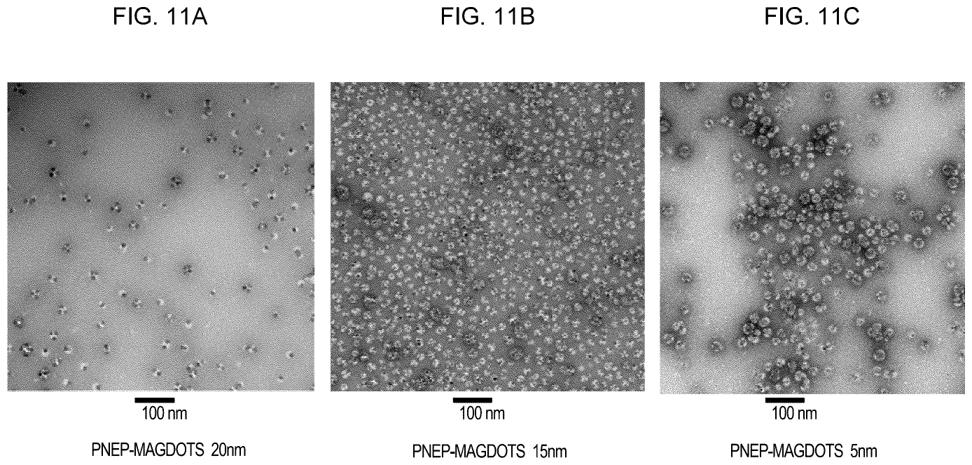

Now, with primary reference to FIGS. 11A through 11c including TEM micrographs illustrating PENP-MagDots (4b) produced by the above method loaded with SPION (2b), and optionally a combination of SPION (2b) and QD (2a) having emission wavelengths in the visible and near infrared spectrum. In the illustrative examples, PENP-MagDots (4b) include one or more QD (2a) having emission wavelengths of 610 nm, in combinations with 20 nm (as shown in the example of FIG. 11A), 15 nm (as shown in the example of 11B) and 5 nm (as shown in the example of FIG. 8C) SPION respectively. The morphology of PENP-MagDots (4b) produced by the above EHD-EM-NP method can be characterized by the use of TEM image and dynamic light scattering of the PENP-MagDots (4b) to determine HD and PDI, as above described.

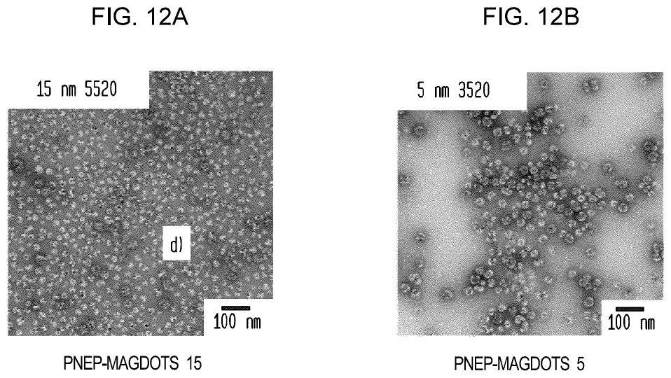

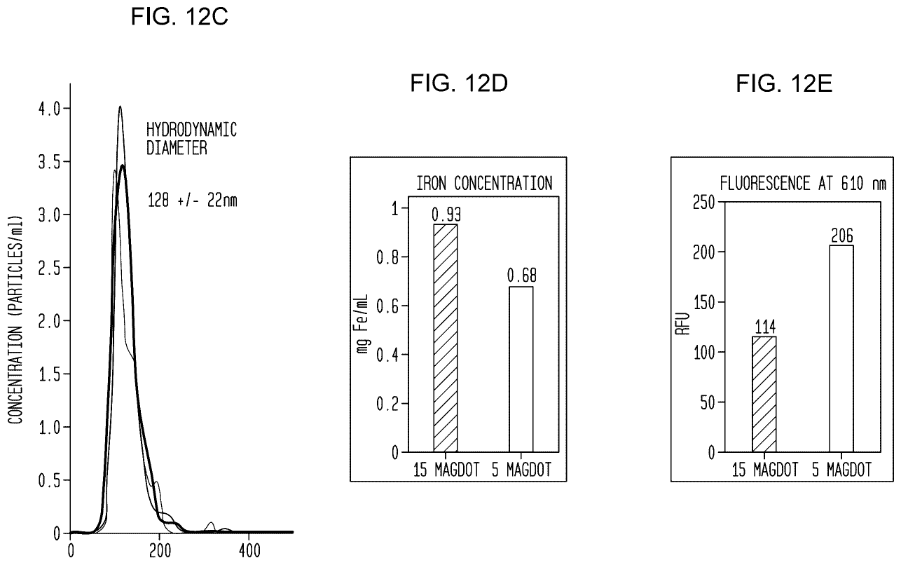

Now, with primary reference to FIGS. 12A and 12B, comprising TEM micrographs illustrating PENP-MagDots (4b) produced in accordance with the above method. DLS can also be used in determining the size of PENP-MagDots (4b). During DLS measurement, a suspension of PENP-MagDots (4b) can be exposed to a light beam and as the incident light impinges on the PENP-MagDots (4b), the direction and intensity of the light beam can be altered due to scattering. PENP-MagDots (4b) in suspension are in constant random motion due to their kinetic energy, the variation of the intensity with time, therefore, contains information on that random motion and can be used to measure the diffusion coefficient of the particles. The HD of the PENP-MagDots R_{H} can be calculated from its diffusion coefficient by the Stokes-Einstein equation D_{f}=k_{B}T/6πηR_{H}, where k_{B} is the Boltzmann constant, T is the temperature of the suspension, and η is the viscosity of the surrounding media.

Now, with primary reference to FIG. 12A, including a TEM micrograph of PENP-MagDots (4b) prepared using a ratio of QD (2a):15 nm SPION (2b):polymer (P) (3) (5:5:20) (“PENP-MagDots15”) and FIG. 12B including a TEM micrographs of PENP-MagDots (4b) prepared using a ratio of QD (2a):5 nm SPION (2b):polymer (3:5:20) (“PENP-MagDots 5”) evidence that the brightness and magnetic susceptibility of the MagDot can be adjusted by loading different sized SPION. The PENP-MagDots15 produced using the ratio 5:5:20 can be more magnetic than the PENP-MagDots 5 using the 3:5:20 ratio. The PENP-MagDots 5 produced using the ratio 3:5:20 can be brighter than the PENP-MagDots 15 using the 5:5:20 ratio.

Now, with primary reference to FIG. 12C, nanoparticle tracking analysis (NTA) of each of the PENP-MagDots 15 shown in the example of FIG. 12A can be illustrated as a plot of particle size versus particle concentration to evidence that the HD of each population can have a uniform size with a PDI of about 0.1 to about 0.2. The determined HD of the PENP-MagDots15 can be about 120 nm to about 140 nm and the determined HD of the PENP-MagDots5 can be about 180 nm to about 220 nm.

Based on the TEM analysis or DLS or NTA analysis, the HD of the PENP-MagDots (4b) produced by the above EHD method can vary depending on the parameters used during EHD and occur within a range of about 40 nm to about 500 nm, and evidence that PENP-MagDots (4b) populations evidence substantially uniform HD and a PDI of about 0.1 to about 0.2.

Now, with primary reference to FIG. 12D comprising a bar graph comparing the iron concentration of PENP-MagDots 5 and PENP-MagDots 15, evidence that PENP-MagDots 15 can be produced having a greater iron concentration than PENP-MagDots 5.

Now, with primary reference to FIG. 12E comprising a bar graph comparing the fluorescence of PENP-MagDots 5 and PENP-MagDots 15, evidence that PENP-MagDots 5 can be produced having a greater fluorescence than PENP-MagDots15.

Thus, by pre-selection of one or more of: SPION (2b) size, QD (2a), PS-b-PEG-NH₂ (3) which can include PS molecular weights ranging from about 1.5 kDa to about 40 kDa and PEG having molecular weights ranging from about 10 kDa to about 40 kDa, and parameters of EHD, and the ratio of QD:SPION:polymer numerous and varied embodiments of the PENP-MagDots (4b) can be produced having utility in a correspondingly numerous clinical and non-clinical applications.

### Preparation of Antibodies.

Again, with primary reference to FIG. 1, the polymer nanomaterial encapsulation system (1) can further include the preparation of antibodies (Ab) (5). In particular embodiments, the PENP (4) produced by FNP, EHD, EM-NPa, self-assembly, or by other means, can be targets for site directed conjugation to antibodies (Ab) (5′). As one illustrative example, half-antibodies can be produced by preferential reduction of the disulfide bonds in the antibody hinge region to yield monovalent components with free thiol groups (“SH”) that can be employed for site-directed conjugation to PENP, PENP-MultiDots, PENP-MagDots or combinations thereof. The reduced antibody fragments can be prepared by reacting 2-mercaptoethylamine hydrochloride (2-MEA), dithiotreitol, mercaptoethanol, or tris(2-carboxyethyl)phosphine (TCEP), to produce a reduced half-antibody as shown in Formula IX.

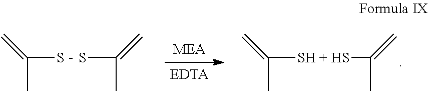

However, this example is not intended to preclude embodiments that may include one or more of: full antibody molecules or their F(ab′)2, Fab′, Fv, rIgF, Fc fragments obtained after pepsin or other enzymatic digestion, or combinations thereof.

A particular illustrative example of a scalable process to produce half-antibodies useful for conjugation to PENP (4), PENP-MultiDots (4a), and/or PENP-MagDots (4b) can include: preparing an antibody stock solution including Immunoglobulin G (“IgG”) (0.5 mg/mL) in a buffer solution of 100 mM phosphate buffer saline (“PBS”) with 10 mM ethylenediaminetetraacetic acid disodium salt dihydrate (“EDTA”) at pH 7.4 (“PBS buffer solution”). An aliquot of 160 μl of the IgG stock solution can be transferred to a first 1.5 mL tube. 2-MEA·HCl having a linear formula of HSCH₂CH₂NH₂·HCl and a molecular weight of 113.60 (CAS NO: 156-57-0) can be obtained from Fisher Scientific PN AAA1437714. In a second 1.5 mL tube, 15 mg of 2-MEA·HCl can be dissolved in 264 μl PBS buffer solution by pipetting. Transfer 5.0 μl of the 2-MEA.HCL solution, respectively, to the 160 μl of the IgG stock solution in the first 1.5 mL tube, vortex, and place 1.5 mL tube containing the IgG solution in a water bath at 37° C. (about 98.6° F.) for 90 min.

The resulting reduced antibody product (also, referred to as “half-antibody product”) (5) may require desalting and a buffer exchange. Desalting removes unreacted MEA contaminants from the half-antibody product while exchanging the half-antibodies into reaction buffer for conjugation to the PENP (4), PENP-MultiDots (4a), and/or PENP-MagDots (4b). An illustrative example of a scalable procedure for desalting and buffer exchange can include utilization of ZEBA™ spin desalting columns obtained from ThermoFisher Scientific, PN 89883, or equivalents thereof. A pair of desalting columns can each be prepared by removing the bottom closure and placing the columns in a 1.5 mL collection tube. Centrifuge at 1500×g for 1 min. to remove storage solution from the desalting column resin. Add 300 μl of 1×100 mM Sodium Phosphate, 1 mM EDTA in water at pH: 6.95-7 (rinse buffer”) or appropriate buffer to the top of the resin bed and centrifuge at 1500 RFC for 1 min. Discard flow-through from collection tube. Repeat addition of 300 μl of 1× rinse buffer two additional times, discarding buffer from the collection tube each time.

The desalting column can now be loaded with the reduced antibody (5) product by placing the first equilibrated desalting column into a first 1.5 mL collection tube marked reduced antibody 1 (“R-Ab-1″), remove the cap from the top of the first desalting column and slowly apply up to a 160 μl reduced antibody product to the center of the compact resin bed. For sample volumes less than 70 μl, apply 15 μl buffer (stacker) to the top of the resin bed after the sample has fully absorbed to ensure maximal protein recovery. Centrifuge at 1500 RCF for 2 min. at 15° C. to 20° C. (59° F. to 68° F.) to collect desalted reduced antibody product. Place the second equilibrated desalting column into a second 1.5 mL collection tube marked reduced antibody 1 (“R-Ab-2”), remove the cap from the top of the second equilibrated desalting column. Transfer the collected reduced antibody (5) product from collection tube R-Ab-1, to the top of the second desalting column. Centrifuge at 1500 RCF for 2 min. at 15° C. to 20° C. (59° F. to 68° F.) to collect desalted reduced antibody product in collection tube R-Ab-2. Cap collection tube R-Ab-2 and place at RT.

### Preparation of Half-Antibody-Polymer Nanocomposite Conjugates.

Now, with primary reference to FIG. 1, the polymer nanomaterial encapsulation system (1) can further include the surface functionalization of PENP (4), PENP-MultiDots (4a), and/or PENP-MagDots (4b) by addition of the reduced antibody product FIG. 1, Block 1 D. As a basis for modification, chemical groups such as amines, carboxylates, thiols, or other reactive groups can be introduced into the PENP (4), PENP-MultiDots (4a), and/or PENP-MagDots (4b) during synthesis, as above described. These groups can be targeted by heterobifunctional chemical cross-linkers (10) including reactive chemical groups connected by spacer arms (as examples: alkane, polyethylene glycol, cleavable disulfide spacers) having various lengths and functions, imparting flexibility, solubility, or other desirable characteristics to the functionalized PENP (4), PENP-MultiDots (4a), and/or PENP-MagDots (4b). As examples, PENP functional group(s) (9) can be reacted with the first reactive group (10′) of the heterobifunctional chemical cross-linkers (10). As illustrative examples, where the functional group (9) of the PENP (4), PENP-MultiDots (4a), and/or PENP-MagDots (4b) comprises an amine, the amine can be reacted with a succinimidyl esters to form amide bonds, or comprises a carboxylate, the carboxylate can be reacted with carbodiimides to produce O-acylisourea intermediates that can be reacted with amines to form amide bonds, or comprise a thiol, the thiol can be reacted with maleimide to form thioether bonds. The reduced antibody (5) product can be reacted with the second reactive group (10″) of the heterobifunctional chemical cross-linker (as shown in the example of FIG. 4C). For example, the second reactive group (10″) of the heterobifunctional chemical cross-linker (10) can comprise a maleimide which can react with the sulfhydryl of the half anti-body (5) to form a thioether bond.

In particular embodiments, both the reactive group associated with the PENP (4) can be modified by reaction with the first reactive group (10a′) of a first heterobifunctional cross-linker (10a) and the reduced antibody (5) product can modified by reaction with the first reactive group (10b′) of a second heterobifunctional chemical cross-linker (10b). The first and second chemical cross-linkers (10a, 10b) can be selected to include second reactive groups (10a″, 10b″) that remain stable in physiological aqueous buffers and upon admixture proceed with fast kinetics and high selectivity to create a covalent bond.

In particular embodiments, the reduced antibody (5) product in collection tube R-Ab-2 can be modified for subsequent conjugation to PENP (4) by reaction of the free sulfhydryl group of the half-antibody (5) with a first reactive group (10a′) comprising a maleimide of a first heterobifunctional cross-linker (10a) to form a thioether bond. In an illustrative example, the first heterobifunctional cross-linker (10a) can comprise sulfo trans-cyclooctene maleimide (“Sulfo-TCO Maleimide”) having a linear formula C₁₇H₂₇N₃O₈S (CAS No. n/a) and a molecular weight of 457.50 g/mol can be obtained from Click Chemistry Tools, PN 1355 comprising Formula X.

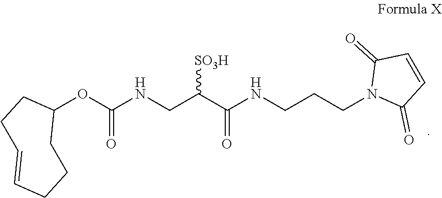 [SMILES: O=C(NCC(C(=O)NCCCN1C(=O)C=CC1=O)S(=O)(=O)O)OC1CC/C=C/CCC1]

Sulfo TCO-Maleimide comprises a water-soluble reagent that enables incorporation of the TCO moiety onto thiol-containing half-antibody. The maleimide group specifically reacts with the sulfhydryl group (“SH”) at pH 6.5 to 7.5 to form a stable thioether bond. The hydrophilic sulfonated spacer arm greatly improves water solubility of Sulfo TCO antibody (“Sulfo-TCO-Ab”) comprising the Formula XI.

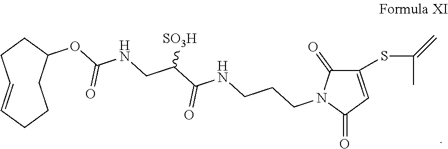 [SMILES: C=C(C)SC1=CC(=O)N(CCCNC(=O)C(CNC(=O)OC2CC/C=C/CCC2)S(=O)(=O)O)C1=O]

An illustrative example of a scalable process to produce the Sulfo-TCO-Ab useful for conjugation to PENP can include one or more of: Remove a tube containing 0.1 mg Sulfo-TCO-maleimide from −20° C. (−4° F.) and bring to RT over 15 min. Add 46.4 μl of anhydrous dimethysufoxide (“DMSO”) having a linear formula C₂H₆OS and a molecular weight of 78.13 g/mol. (CAS No. 67-68-5) to 0.1 mg Sulfo-TCO-maleimide. Vortex the Sulfo-TCO-maleimide in the tube for 10 min. to ensure dissolution in the DMSO. Repeatedly mix the Sulfo-TCO-maleimide in DMSO via pipette set to 60 μl. Add 5 μl or 10 μl of Sulfo-TCO-maleimide to the R-Ab-2 tube containing the reduced antibody (5). Discard the remaining Sulfo-TCO-maleimide. Relabel the tube containing the Sulfo-TCO-Ab. Cap and vortex the tube containing the Sulfo-TCO-Ab. Place the Sulfo-TCO-Ab tube at 4° C. (39.2° F.) for 20-24 hr.

The PENP (4) products above described can be reacted with a first reactive group of a second heterobifunctional cross-linker. The modified PENP (4) can then be associated with one or more target moieties (24), including one or more of: cells, cell organelles, proteins, peptides, amino acids, oligonucleotides, ligands, or linkers by selective reaction of the second reactive group with the corresponding function groups within the target moiety (24).

In particular embodiments, the second heterobifunctional cross-linker (10b) can comprise methyltetrazine polyethylene glycol-4-N-hydroxysuccinimide ester (“TZ-PEG₄-NHS ester”) having a linear formula C₂₄H₃₁N₅O₉ having a molecular weight of 533.53 g/mol (CAS NO. 182907-92-1) comprising Formula XII.

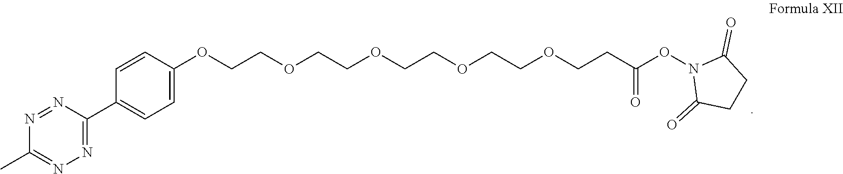 [SMILES: Cc1nnc(-c2ccc(OCCOCCOCCOCCOCCC(=O)ON3C(=O)CCC3=O)cc2)nn1]

An illustrative example, the TZ-PEG₄-NHS ester comprising Formula XIII can be reacted with the functional group (9) of the multi-arm PEG-NH₂ (3) comprising multiple amine as shown in Formula IV or Formula V of the PENP (4) to produce PENP-PEG₄-TZ comprising Formula XIII.

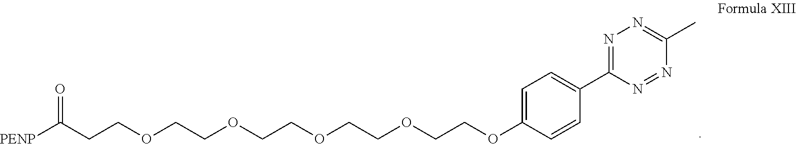

As one example, a scalable process to produce the PENP-PEG₄-TZ of Formula XIII using the PENP (4) functional group (9) comprising an amine of the multi-arm PEG-NH₂ (3) shown in Formula IV or Formula V can include one or more of: bring 1 mg of TZ-PEG-NHS ester to RT over approximately 15 min. Transfer 93.7 μl or 375 μl of anhydrous DMSO to TZ-PEG₄-NHS ester to produce 20 mM or 5 mM TZ-PEG₄-NHS in DMSO. Mix TZ-PEG₄-NHS ester in DMSO by pipette for complete dissolution of TZ-PEG₄-NHS ester in DMSO. Label 1.5 mL tube as PENP-Wavelength-TZ. Transfer PENP into PENP-Wavelength-TZ tube (120 μl of PENP-MagDots or 80 μl of PENP-MultiDots). In PENP-MultiDots (4a) embodiments about 30 nmol of amine can be activated by reaction with about 70 nmol of TZ-PEG₄-NHS ester and in PENP-MagDot (4 b) embodiments about 120 nmol of amine can be activated by reaction with 70 nmol of TZ-PEG₄-NHS ester. Pipette 3.5 μL of 20 mM TZ-PEG₄-NHS ester into the PENP-MultiDot-Wavelength-TZ tube or 15 μl of 5 mM TZ-PEG₄-NHS ester into the PENP-MagDot-Wavelength-TZ tube. Mix thoroughly with an air displacement pipette. React at RT for 2 hrs.

In particular embodiments the PENP-PEG₄-TZ comprising Formula XIII can be reacted with Sulfo-TCO-Ab comprising the Formula XI in an inverse-electron demand Diels-Alder [4+2] cycloaddition reaction of TCO with TZ to form a dihydropyridazine bond to produce conjugate PENP-Antibodies (6) (“PENP-Ab”) comprising Formula XIV including but not limited to PENP-MultiDots (4a) shown in the example of FIG. 4A or PENP-MagDots (4b) as shown in the example of FIG. 4B, where R₁ comprises PENP and where R₂ comprises an Ab.

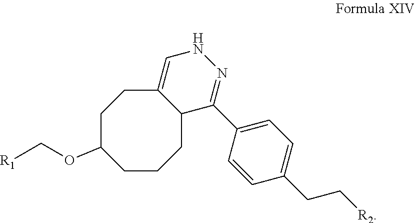

As one example, a scalable process to produce the PENP-Ab (6) of Formula XIV using PENP-PEG₄-TZ (PENP-MagDots-PEG₄-TZ or PENP-MultiDots-PEG₄-TZ) and Sulfo-TCO-Ab can include a pair of desalting columns (as a non-limiting example Zeba™ desalting columns) each prepared by removing the bottom closure and placing each of the pair of desalting columns in a 1.5 mL collection tube. Centrifuge at 1500×g from 1 min. to remove storage solution from the desalting column resin. Add 300 μL of 1× rinse buffer or appropriate buffer to the top of the resin bed and centrifuge at 1500 RFC for 1 minute. Discard flow-through from the respective collection tubes. Repeat addition of 300 μL of 1× rinse buffer (pH 6.0) two additional times, discarding buffer from the respective collection tubes each time. Transfer the PENP-PEG₄-TZ to the first of the pair of desalting columns. Transfer the Sulfo-TCO-Ab to the second of the pair of desalting columns. Centrifuge the first and second desalting columns at 1500 RCF for 2 min. at 15° C. to 20° C. (59° F. to 68° F.) to collect PENP-PEG₄-TZ and Sulfo-TCO-Ab products post excess unconjugated linker removal. Mix the desalted PENP-PEG4-TZ (120 μL PENP-MagDots or 80 uL PENP-MultiDot) in the first collection tube by pipette. Transfer the desalted 160 μL (80 μg) Sulfo-TCO-Ab from the second collection tube into the PENP-PEG4-TZ in the first collection tube. Let react for 24 hrs. at 4° C. (39.2° F.) to produce PENP-Ab (PENP-MagDots-Ab or PENP-MultiDots-Ab).

Subsequent to conjugation, the resulting PENP-Ab (6) product can be purified. As one example, a scalable size exclusion process to purify PENP-MultiDots-Ab can include: label a 1.5 mL microcentrifuge tube as “PENP-MultiDots-Ab-Wavelength”. Introduce 3 mL of gel filtration resin (e.g. Sephacryl® available from SigmaAldrich, PN S400HR) into 3 mL borate buffer saline (50 mM sodium borate, 100 mM sodium phosphate, 7.3-7.5 pH (“borate buffer”)) (“BBS”). Transfer the suspension of gel filtration resin into a size exclusion column (“SEC”). Slowly pipette PENP-MultiDots-Ab (6a) solution to the sides of the SEC. Connect an air syringe to the SEC. Push 1 mL of air into the SEC via the air syringe. Allow the PENP-MultiDots-Ab (6a) solution to move into the gel filtration resin. Remove air syringe from the SEC. Pipette 200 μl BBS buffer to the sides of the SEC. Reconnect the air syringe to the SEC. Push 1 mL of air into the SEC via the air syringe. Allow the PENP-MultiDots-Ab (6a) solution to move into SEC packing. Remove the air syringe from SEC. Pipette 1000 μl BBS to the sides of the SEC. Reconnect the air syringe to the SEC. Reduce ambient light incident upon the SEC. Direct ultraviolet light (“UV light”) incident upon SEC. Push 1 mL of air into the SEC via the air syringe. Begin collection of BBS eluent from SEC. Upon detection of first fluorescent drop from SEC, collect fluorescent drops 2 through 5 in PENP-MultiDots-Ab-Wavelength collection tube. Mix collected fluorescent drops sample with a pipette. Remove the air syringe, dispose the gel filtration resin and clean the SEC.

As one example, a scalable size exclusion process to purify PENP-MagDots-Ab (6b) can include: mix PENP-MagDots-Ab (6b) in conjugation buffer thoroughly with an air displacement pipette. Place a MPSC in a magnetic field gradient (e.g. up to about 100-200 T/m). Introduce the PENP-MagDots-Ab (6b) in conjugation buffer into the MPSC. Allow the conjugation buffer to pass through the MPSC. Transfer 300 μL of BBS into MPSC. Pipette to mix PENP-MagDots-Ab in reaction buffer in the MPSC. Allow the reaction buffer to pass through the MPSC. Transfer 300 μL of reaction buffer into the MPSC. Pipette to mix PENP-MagDots-Ab (6b) in reaction buffer in the MPSC. Allow the reaction buffer to pass through the MPSC. Remove any reaction buffer from tip of the MPSC. Extract the MPSC from the magnetic field gradient. Once the MPSC is extracted from the magnetic field gradient, the MPSC can no longer retain the PENP-MagDots-Ab (6b) and the PENP-MagDots-Ab (6b) can be eluted. Place the MPSC in a PENP-MagDots-Ab (6b) collection tube labeled PENP-MagDots-Ab-. Transfer 200 μl of reaction buffer into the MPSC. Pipette to mix PENP-MagDots-Ab (6b) in reaction buffer in the MPSC. Collect PENP-MagDots-Ab (6b) into the MagD-Antibody-Date collection tube. Mix collected PENP-MagDots-Ab (6b) thoroughly with an air displacement pipette.

### Cell Labelling and Flow Cytometry Utilizing PENP-MultiDots-Ab.

PENP-MultiDots-Ab (6a) can effectively and specifically label target moieties (24) comprising cellular targets (24′). Single particle imaging, cellular imaging, or flow cytometry using PENP-MultiDots-Ab (6a) labeled cellular targets (24′) evidence very high fluorescence intensity compared to labeling cellular targets (24′) with conventional dyes. The PENP-MultiDots-Ab (6a) can effectively and specifically label cell surface receptors and subcellular structures in both live or fixed cells without any detectable non-specific binding. Flow cytometry can be performed to evaluate PENP-MultiDots-Ab (6a) performance and to show the high cellular label brightness compared to conventional dyes and quantum dot probes.

As one illustrative example of PENP-MultiDots-Ab (6a) labeling of cellular targets (24′), peripheral blood mononuclear cells (“PBMC”) (24′) can be labeled using PENP-MultiDots-Ab (4a). A scalable method of labeling PBMC with PENP-MultiDots-Ab (6a) can include one or more of: diluting peripheral blood as an iso-osmatic solution 50:50 with Hank's balanced salt solution (“HBSS”) (SigmaAldrich, PN H6648). Diluted peripheral blood can be layered over a Ficoll-Hypaque gradient (density=1.077 g/cm³) and centrifuged at 1350 RPM for 30 min. without brake. Serum can be aspirated and discarded. PBMC can be removed and transferred to another collection tube and washed with PBS. The PBMC pellet can be resuspended in 10 mL PBS and cell counts performed on a hemacytometer. A million PBMC (24′) in 200 μl of PBS can be placed in a 12×75 mm flow cytometry tube to which 10 μl of PENP-MultiDots-mouse anti human CD3 (6a) having a 610 nm emission wavelength was added. PBMC (24′) and PENP-MultiDots-mouse anti human CD3 (6a) incubated for 25 min. at RT. After expiration of the incubation period, 500 μl of additional PBS can be added to the PENP-MultiDots-mouse anti human CD3 bound PBMC (7) (“PENP-MultiDots-mouse anti human CD3-PBMC”). The PENP-MultiDots-mouse anti human CD3-PBMC (7) were centrifuged for 7 min. at 1800 RPM at RT. The supernatant can be discarded and the PBMC/PENP-MultiDots-mouse anti human CD3-PBMC (7) can be re-suspended in 400 μl of PBS for flow cytometry analysis (8) for detection of PENP-MultiDots-mouse anti human CD3-PBMC (7) population using a Cytek Northern Lights spectral flow cytometer. The CD3 protein complex can be an important T cell marker for the classification of malignant lymphomas and leukemias (T cell neoplasms). CD3 can also be used for the identification of T cells in coeliac disease, lymphocytic colitis and collagenous colitis.

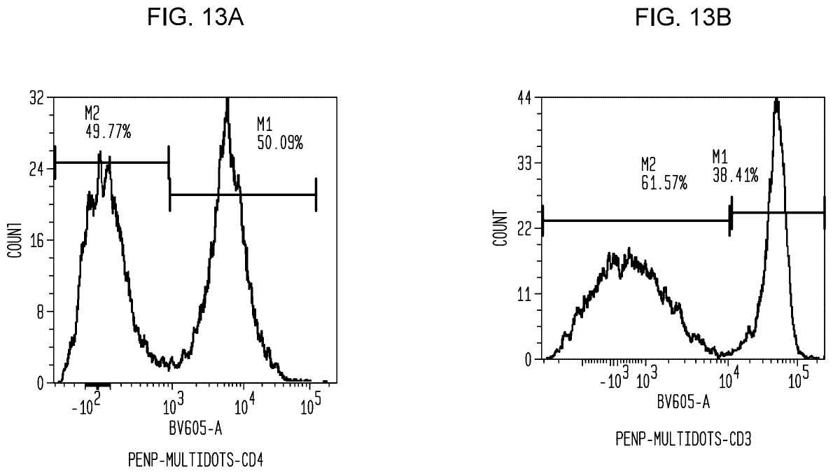

Now, with primary reference to FIG. 13A, the univariant histogram (florescence intensity versus particle number) depicts detection of PENP-MultiDots-mouse anti human CD4-PBMC (7) population (“Peak M1”) in a sample of PBMC/PENP-MultiDots-mouse anti human CD4-PBMC (7) obtained by the method above described. The histogram evidences that 38.41% of the cells in the sample can be identified as the PENP-MultiDots-mouse anti human CD4-PBMC (7) population within the sample. In particular embodiments, the PENP-MultiDots-mouse anti human CD4-PBMC (7) population can be sorted and isolated for further analysis.

Now, with primary reference to FIG. 13B, the univariant histogram (florescence intensity versus particle number) depicts detection of PENP-MultiDots-mouse anti human CD3-PBMC (7) population (“Peak M1”) prepared in accordance with the methods above described. The histogram evidences that 50.09% of the cells in the sample can be identified as the PENP-MultiDots-mouse anti human CD3-PBMC (7) population within the sample.

PENP-MagDots-Ab (6b) can also effectively and specifically label cellular targets (24′). Single particle imaging, cellular imaging, or flow cytometry analysis (8) using PENP-MagDots-Ab (6b) labeled cellular targets (24′) evidence very high fluorescence intensity compared to labeling cellular targets (24′) with conventional dyes or quantum dots. The PENP-MagDots-Ab (6b) can effectively and specifically label cell surface receptors and subcellular structures in both live or fixed cells without any detectable non-specific binding. Flow cytometry (8) can be performed to evaluate PENP-MagDots-Ab (6b) performance and to show the high cellular label brightness compared to conventional dyes and quantum dot probes.

As one illustrative example of PENP-MagDots-Ab (6b) labeling of cellular targets (24′), peripheral blood mononuclear cells (“PBMC”) (24′) can be labeled using PENP-MagDots-Ab (6b). As one example, a scalable method can include one or more of: diluting peripheral blood as an iso-osmatic solution 50:50 with Hank's balanced salt solution (“HBSS”) (SigmaAldrich, PN H6648). Diluted peripheral blood can be layered over a Ficoll-Hypaque gradient (density=1.077 g/cm³) and centrifuged at 1350 RPM for 30 min. without brake. Serum can be aspirated and discarded. PBMC can be removed and transferred to a PBMC collection tube and washed with PBS. The PBMC pellet can be resuspended in 10 mL PBS and cell counts performed on a hemacytometer. A million PBMC (7) in 200 μl of PBS can be introduced into a 12×75 mm flow cytometry tube to which 20 μL of PENP-MagDots-mouse anti human CD3 (6b) having a 610 emission spectrum were added. PBMC and PENP-MagDots-mouse anti human CD3 (6b) are incubated for 25 min. at RT. After expiration of the incubation period, 200 μl of additional PBS can be added to the PENP-MagDots-mouse anti human CD3 bound PBMC (7) (“PENP-MagDots-mouse anti human CD3-PBMC”) in the flow cytometry tube. Place the flow cytometry tube containing PENP-MagDots-mouse anti human CD3-PBMC (7) in PBS in a magnetic field gradient (23) (e.g. up to about 100-200 T/m). After expiration of 15 min., the nonmagnetic fraction can be aspirated from the flow cytometry tube and placed a nonmagnetic fraction collection tube. The flow cytometry tube containing the magnetically retained PENP-MagDots-mouse anti human CD3-PBMC (7) can be removed from the magnetic field gradient and the PENP-MagDots-mouse anti human CD3-PBMC (7) can be resuspended in 400 μl PBS. The PBMC/PENP-MagDots-mouse anti human CD3 reaction solution prior to magnetic separation, the nonmagnetic fraction after magnetic separation, and the PENP-MagDots-mouse anti human CD3-PBMC fraction after magnetic separation, were each analyzed by flow cytometer (8).

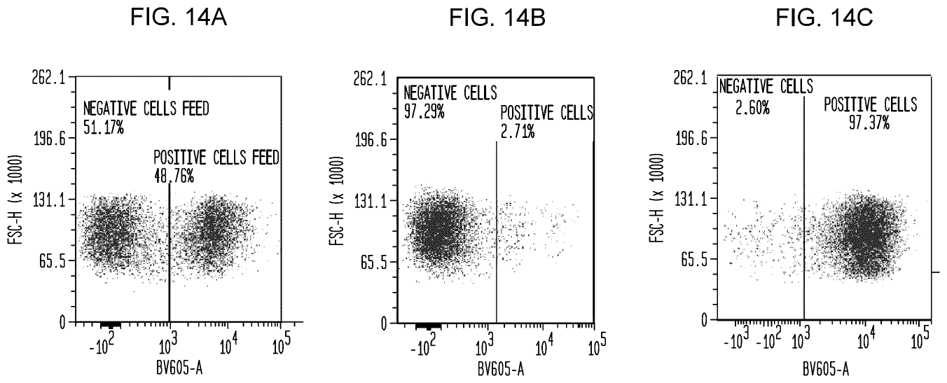

Now, referring primarily to FIGS. 14A through 14C, which depict bivariant dot plots (forward scatter area versus forward scatter height) obtained by flow cytometry analysis (8) of each of the PBMC/PENP-MagDots-mouse anti human CD3 reaction solution prior to magnetic separation (as shown by the example of FIG. 14A), the non-magnetic fraction after magnetic separation (as shown by the example of FIG. 14B), and the PENP-MagDots-mouse anti human CD3-PBMC fraction after magnetic separation (as shown by the example of FIG. 11C). The flow cytometry dot plots evidence the proportion of CD3 negative cells to CD3 positive cells. As evidenced by FIG. 14A, the PBMC/PENP-MagDots-mouse anti human CD3 reaction solution prior to magnetic separation contains 51.17% CD3 negative cells and 48.76% CD3 positive cells. FIGS. 14B and 14C, evidence that after magnetic separation the non-magnetic fraction contains largely CD3 negative cells 97.29% and very few CD3 positive cells 2.71%, whereas by contrast, FIG. 11C evidence that the PENP-MagDots-mouse anti human CD3-PBMC fraction after magnetic separation includes very few CD3 negative cells 2.60% and largely CD3 positive cells 97.37%. This evidences the substantial advantage of using PENP-MagDots-Ab (6b) to capture and purify cellular targets (24′).

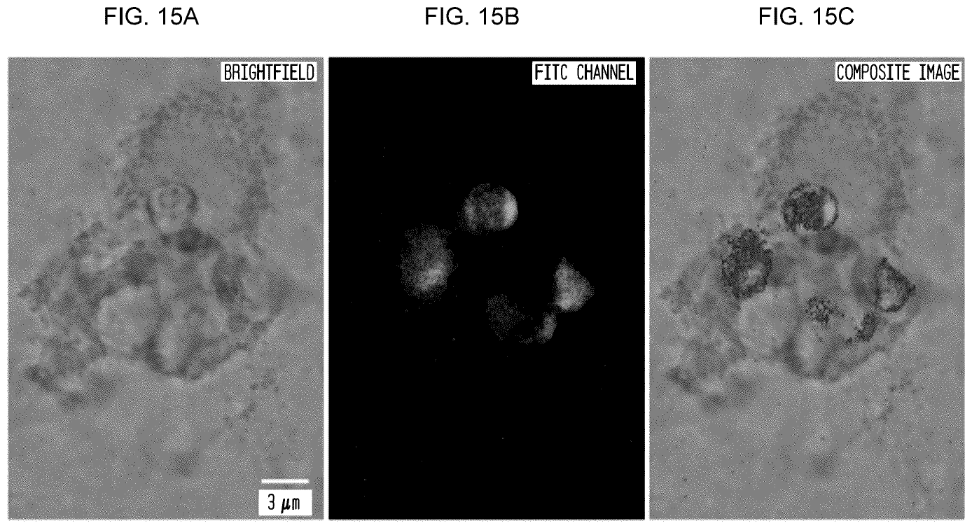

Now, referring primarily to FIGS. 15A through 15C which illustrate labeling of Hawaiian Bobtail Squid (Euprymna scolopes) hemocytes with PENP-MagDots. Adult Bobtail Squid can be incubated for 3 hr. with 100 uL of PENP-MagDots containing a QD having an emission wavelength of 610 nm. The images evidence that Hawaiian Bobtail Squid (Euprymna scolopes) hemocytes passively uptake PENP-MagDots 610 nm.

### Quantum Dot Stacks In PENP-MultiDots-Ab Labeled Cells.

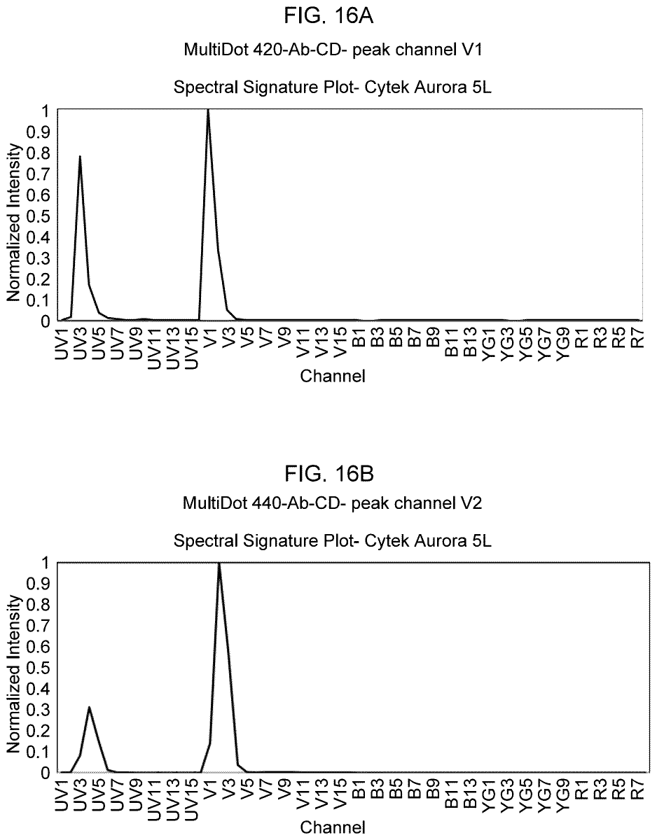

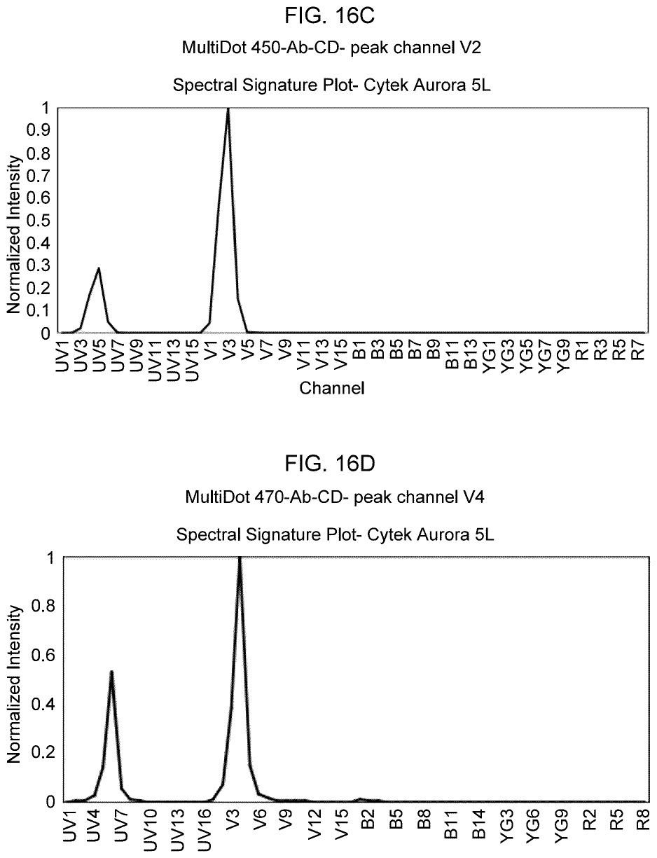

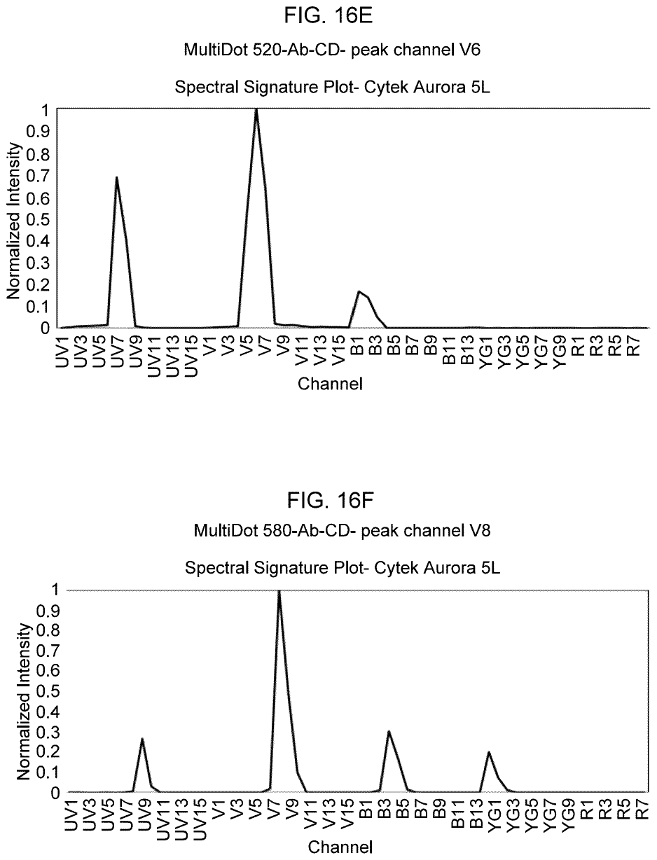

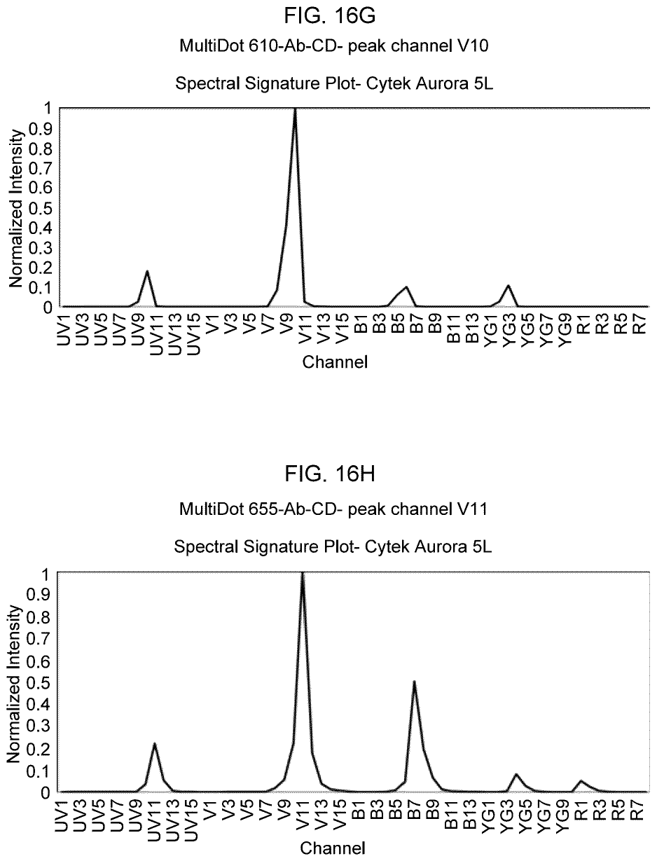

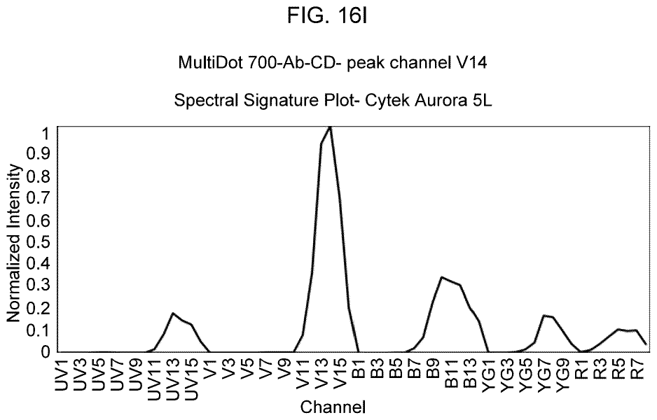

Now, with primary reference to FIGS. 16A through 16I which provide the emission spectrum histograms of set of PENP-MultiDots-mouse anti human CD4 including QD (2a) having peak emissions at 420 nm, 440 nm, 450 nm, 470 nm, 520 nm, 580 nm, 610 nm 655 nm, and 700 nm, each made in accordance with the method of FIG. 1, Blocks 1A through 1E, obtained by operation of a high-performance spectral flow cytometer that captures the entire fluorescence emission spectrum from each fluorophore using four or five lasers and 48-64 detection channels. In the illustrative example of FIGS. 16A through 16I the emission spectrum graphs were obtained using a CYTEK AURORA® spectral flow cytometry available from Cytek Biosciences, Fremont, California.

CytoCompcells® obtained from Beckman Coulter were resuspended to 1 ml with provided serum. Cells were pipetted up and down to create a single cell suspension. To the resuspended cells, 10 ml of PBS buffer+2 mM EDTA+5% BSA (“FACS buffer”) was added, and cells were centrifuged at 1800 RPMs for 7 minutes. Serum and FACS buffer were aspirated off the cell pellet and cells were resuspended in 1 ml FACS buffer. Aliquots in the amount of 100 μl were taken from the washed resuspended cells and placed into 10 flow cytometry tubes (12×75 mm) and labeled as unlabeled, 420 nm, 450 nm, 470 nm, 520 nm, 580 nm, 610 nm, 655 nm, and 700 nm respectively. To each of the tubes except for the unlabeled control, 10 μl of PNEP-Multidots-Ab CD4 were added to the appropriately labeled tube. Cells and MultiDots-Ab were incubated at room temperature for 30 minutes and then washed with 2× the staining volume with FACS buffer. The supernatant was aspirated and the cells were resuspended in 250 μl of FACS buffer per tube and immediately run on the CYTEK AURORA® spectral flow cytometry generating the histograms depicted in FIGS. 16A through 16 I.

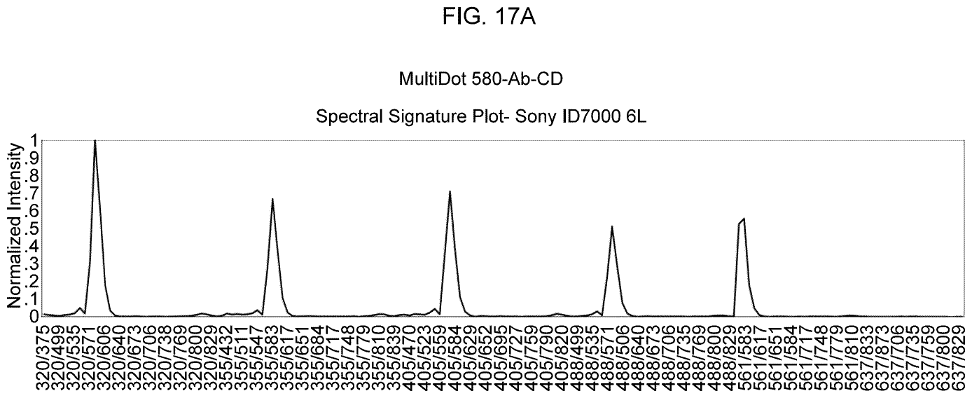

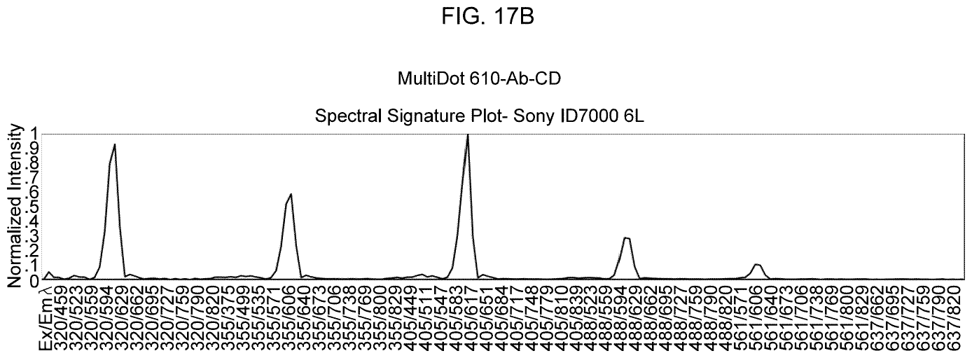

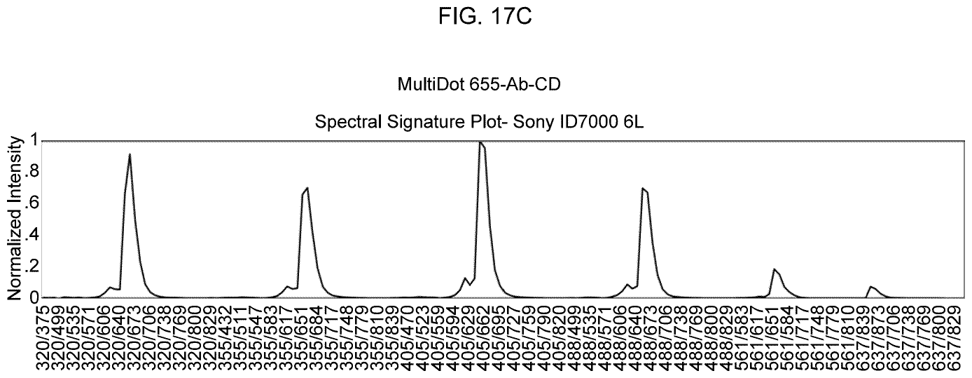

Now, with primary reference to FIGS. 17A through 17C which provide emission spectrum histograms of a set of PENP-MultiDots-mouse anti human CD4 including QD (2a) having peak emissions at 580 nm, 610 nm, and 655 nm, each made in accordance with the method of FIG. 1, Blocks 1A through 1E obtained by operation of a spectral flow cytometer that captures the entire fluorescence emission spectrum for each fluorophore using up to 7 lasers and up to 186 detection channels. In the illustrative example of FIGS. 17A through 17C the emission spectrum graphs were obtained using a SONY® ID7000 Spectral Flow Cytometer available from Sony Biotechnology, San Jose, California.

CytoCompcells® obtained from Beckman Coulter were resuspended to 1 ml with provided serum. Cells were pipetted up and down to create a single cell suspension. To the resuspended cells, 10 ml of PBS buffer+2 mM EDTA+5% BSA (FACS buffer) was added, and cells were centrifuged at 1800 RPMs for 7 minutes. Serum and buffer were aspirated off the cell pellet and cells were resuspended in 400 μl FACS buffer. Aliquots in the amount of 100 μl were taken from the washed resuspended cells and placed into 4 flow cytometry tubes (12×75 mm) and labeled as unlabeled, 580 nm, 610 nm, and 655 nm. To each of the tubes except for the unlabeled control, 10 μl of Multidots-Ab conjugated to the CD4 antibody were added to the appropriately labeled tube. Cells and MultiDots-Ab were incubated at room temperature for 30 minutes and then washed with 2× the staining volume with FACS buffer. The supernatant was aspirated and the cells were resuspended in 250 μl of FACS buffer per tube and immediately run on the Sony ID7000 Spectral cytometer instrument generating the histograms seen in FIGS. 17A through 17C.

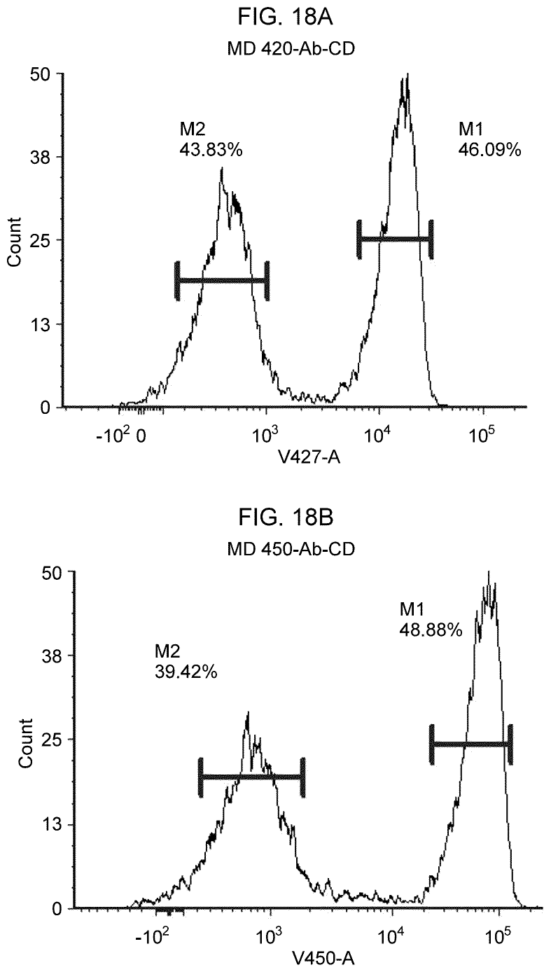

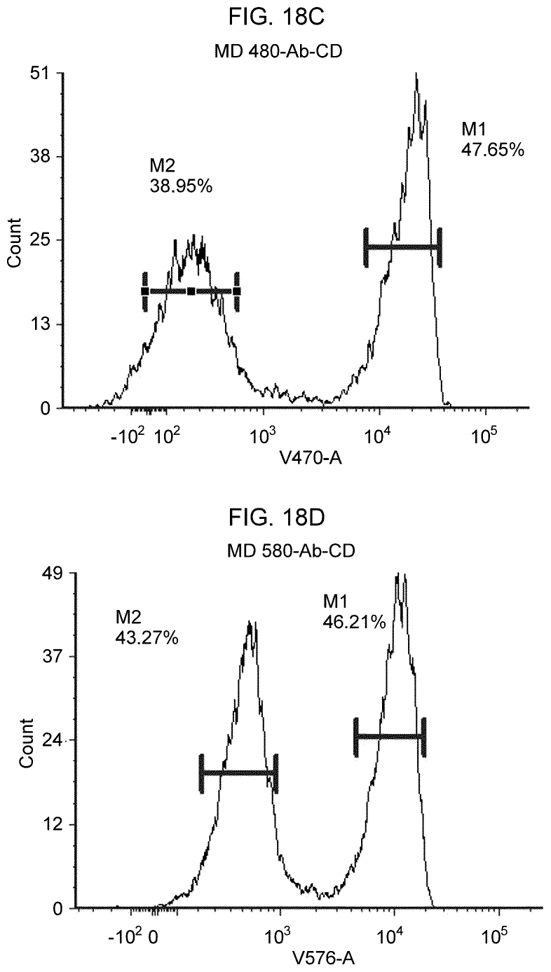

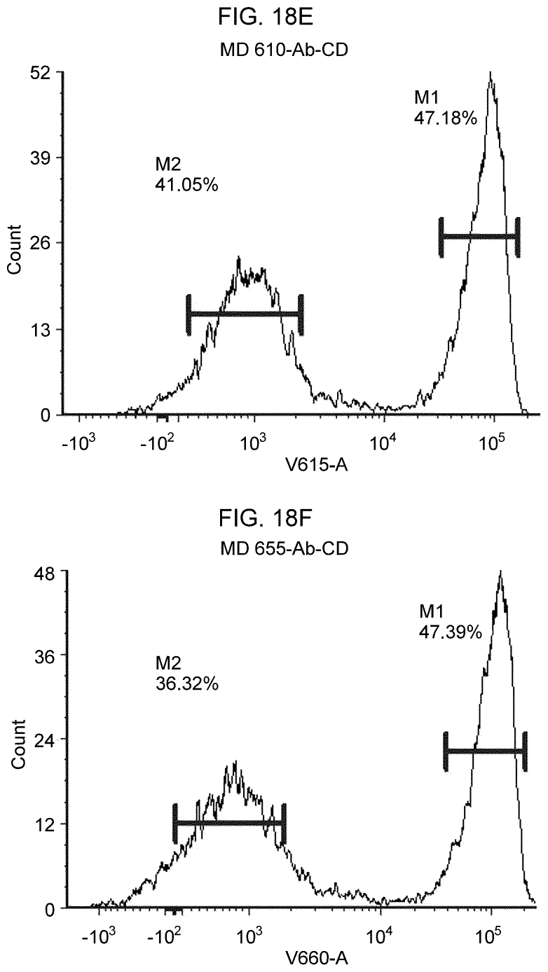

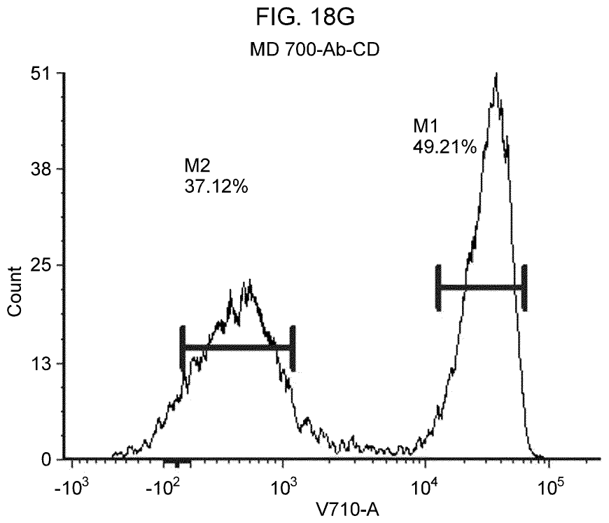

Now, with primary reference to FIGS. 18A through 18G, which provide histograms of a set of PENP-MultiDots-mouse anti human CD4 including QD (2a) having peak emissions at 420 nm, 450 nm, 480 nm, 580 nm, 610 nm, 655 nm, and 700 nm, each made in accordance with the method of FIG. 1, Blocks 1A and 1B obtained by operation of a spectral flow cytometer that captures the entire fluorescence emission spectrum for each fluorophore using up to 5 lasers and up to 48 detection channels. In the illustrative example of FIGS. 18A through 18G the histograms of PNEP MultiDots were obtained using a BD FACSymphony® A5 SE available from Becton Dickinson, Qume Drive, San Jose, California.

CytoCompcells® obtained from Beckman Coulter were resuspended to 1 ml with provided serum. Cells were pipetted up and down to create a single cell suspension. To the resuspended cells, 10 ml of PBS buffer+2 mM EDTA+5% BSA (FACS buffer) was added, and cells were centrifuged at 1800 RPMs for 7 minutes. Serum and buffer were aspirated off the cell pellet and cells were resuspended in 800 μl FACS buffer. Aliquots in the amount of 100 μl were taken from the washed resuspended cells and placed into 8 flow cytometry tubes (12×75 mm) and labeled as unlabeled, 420 nm, 450 nm, 470 nm, 610 nm, 655 nm and 700 nm. To each of the tubes except for the unlabeled control, 10 μl of Multidots-Ab conjugated to the CD4 antibody were added to the appropriately labeled tube. Cells and MultiDots-Ab were incubated at room temperature for 30 minutes and then washed with 2× the staining volume with FACS buffer. The supernatant was aspirated and the cells were fixed with 4% formalin sent chilled to Cincinnati Children's Hospital to be run on their Becton Dickinson A5 Spectral cytometer instrument generating the histograms seen in FIGS. 18A through 18G.

As evidenced by the examples of FIGS. 16A through 16I, 17A through 17C, and 18A through 18G, from instrument to instrument, no matter the strength of the laser and the difference in the number of channels, the emission spectra from each of the PNEP-Multidots-Ab CD4 have narrow spectral emissions with an average full width of half maximum of 20 nm.

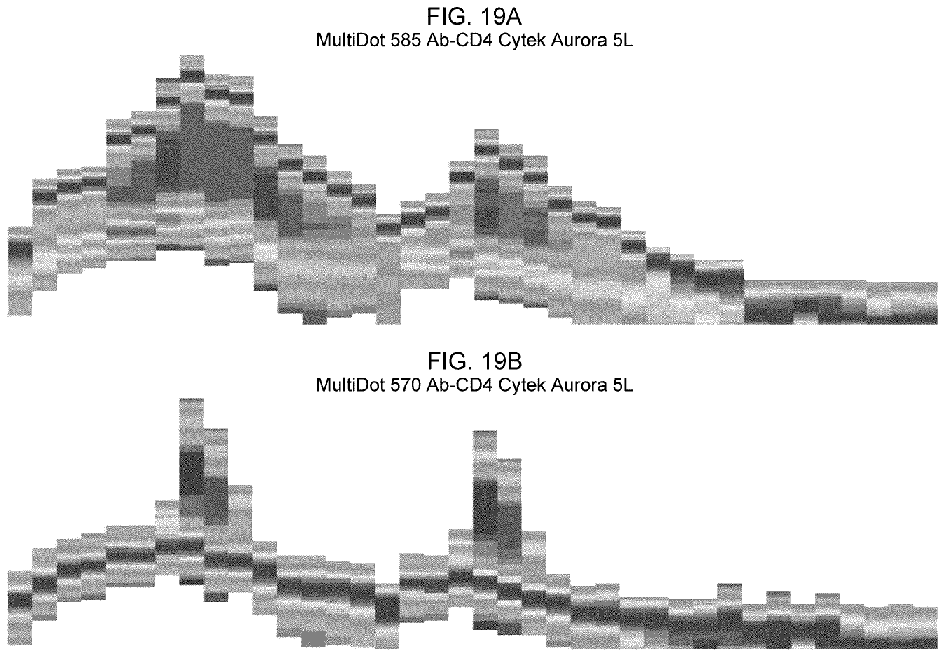

Now, with primary reference to FIGS. 19A and 19B, which provide an emission profile of PNEP MultiDot 585-Ab CD4 made in accordance with the method of FIG. 1, Blocks 1A through 1E as compared to an emission profile of an organic dye Starbright Violet 570 obtained from BioRad Laboratories, Hercules, California. The emission profiles were obtained by operation of a high-performance spectral flow cytometer that captures the entire fluorescence emission spectrum from each fluorophore using four or five lasers and 48-64 detection channels. In the illustrative example of FIGS. 9A and 19B, the emission profiles were obtained using a CYTEK AURORA® spectral flow cytometry available from Cytek Biosciences, Fremont, California.

By way of this example, organic polymer dyes tend to have wider or broader emissions off more than one laser excitation source. While this may be a benefit in conventional flow cytometry as the broader signal allows for the fluorophore to be detected in each filter due to decompensation between dyes. With advent of spectral unmixing, the spectral signatures of each of these dyes are also unique with similar emission colors, which leads to panel design with overlapping emissions that used to be a problem in conventional flow cytometry. These similar emissions from organic and polymer dyes might be able to be used in combination in spectral flow cytometry now, but still impact the spillover spectral matrix data on the design of a fluoresce.

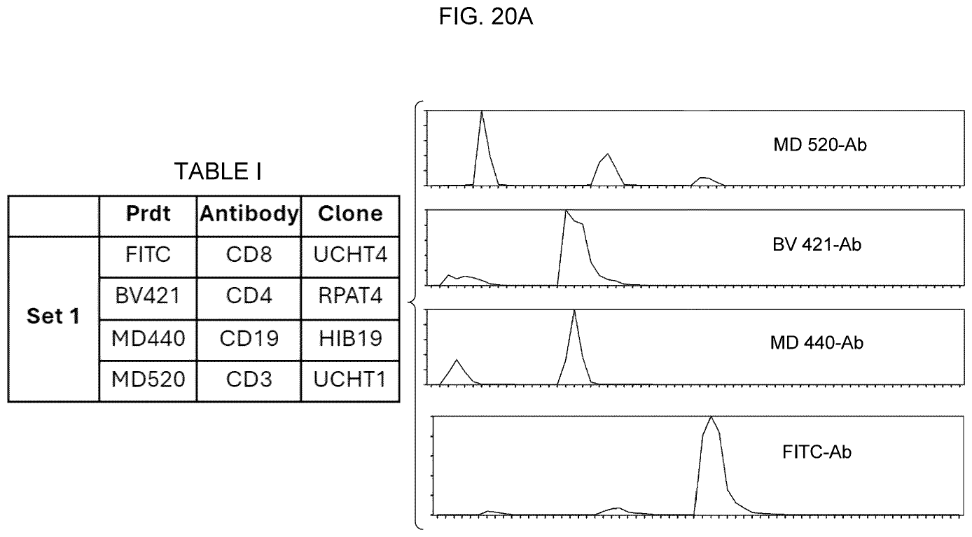

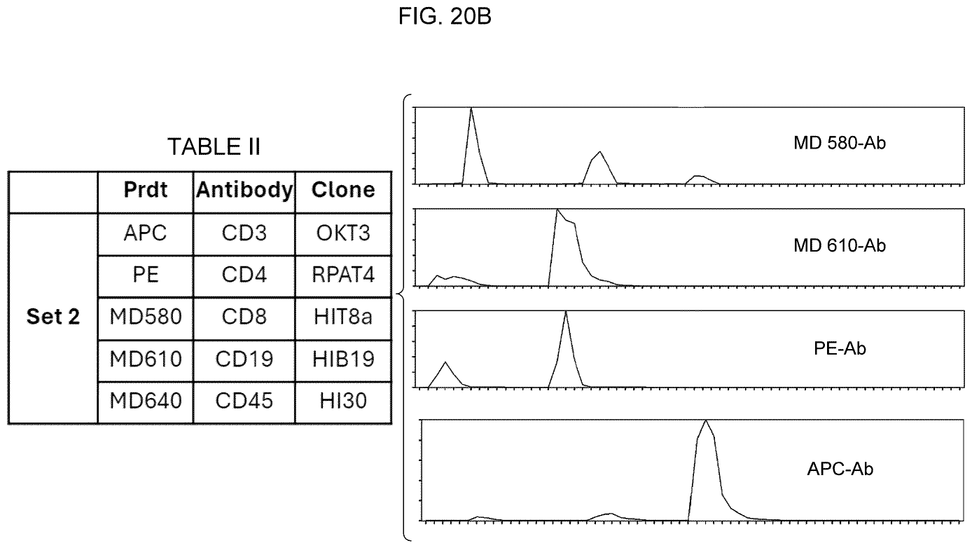

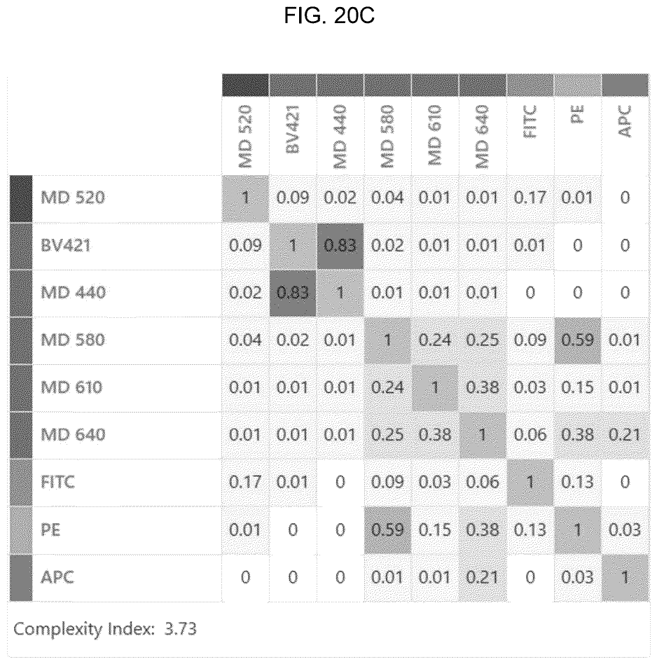

Now, with primary reference to FIGS. 20A through 20C, as further evidence of the unique and narrow spectral signature of PNEP-MultiDots-Ab CDs made in accordance with the method of FIG. 1, Blocks 1A through 1E, PNEP-MultiDots-Ab CDs were compared to similar emission fluorophores that excite off different lasers and emit in different channels (as shown in FIGS. 20A and 20B) to yield a spectral flow cytometry similarity index (as shown in FIG. 20C) with a metric ranging from 0 to 1 that quantifies how closely the full emission spectra of two fluorophores match, with 1 being identical and 0 being unique. The metric assists in determining whether fluorophores can be used together in a panel, where lower values indicate better spectral separation. In the illustrative example, the single stained controls and the multiplexed samples were analyzed using a CYTEK AURORA® spectral flow cytometry available from Cytek Biosciences, Fremont, California.

Now, with primary reference to FIG. 20A, Table I, which indicates the single stained controls analyzed for each color prior to analysis of the multiplexed samples. Referring to Table I which identifies the PNEP-MultiDots and associated Abs and fluorophores and the associated Abs in a first set of PNEP-MultiDots-Ab and fluorophores Ab (“Set 1”). Set 1 includes MultiDot 440-Ab CD19, MultiDot 520-Ab CD3, Brilliant Violet 421 CD4 and fluorescein isothiocyanate CD8.

Now, with primary reference to FIG. 20B, Table II, which indicates the single stained controls analyzed for each color prior to analysis of the multiplexed samples. Referring to Table II which identifies the PNEP-MultiDots-Ab and fluorophores Ab (“Set 2”). Set 2 includes MultiDot 580-Ab CD8, MultiDot 610-Ab CD19, MulitDot 640-Ab CD45, allophycocyanin CD3, and phycoerythrin CD4.

FIG. 20C provides a similarity index for MultiDot and fluorophore multiplex samples analyzed by spectral flow cytometry. Unmixing was performed after running the single stained controls. The similarity index indicates more difficult unmixing between the BV421 and the MD440, but still under 0.9 which is considered the level at which unmixing would be problematic. The rest of the combinations resulted in similarity complexes between 0.01 and 0.4, thereby showing promise of use of MultiDots with fluorophores that have similar emission excited off different lasers.

While the similarity index can aid in multiplexed panel design, it does not indicate the spread that one dye can contribute to another fluorophore known as the spillover spread matrix. While compensation can remove the mean signal spillover, but it does not remove the variance, which presents as noise in secondary detectors. That noise can be calculated and expressed in spillover spread matrix. The spillover spread matrix (“SSM”) in flow cytometry quantifies how much signal from one fluorochrome spreads into the detector of another, independent of detector voltage or fluorochrome brightness. With values ≤5 generally acceptable, 5-9 questionable, and ≥10 undesirable. The CYTEK AURORA® spectral flow cytometry and other flow cytometers can determine SSM of a multiplexed panel. A number greater than 5 indicates impactful spread of that certain fluorophore combination.

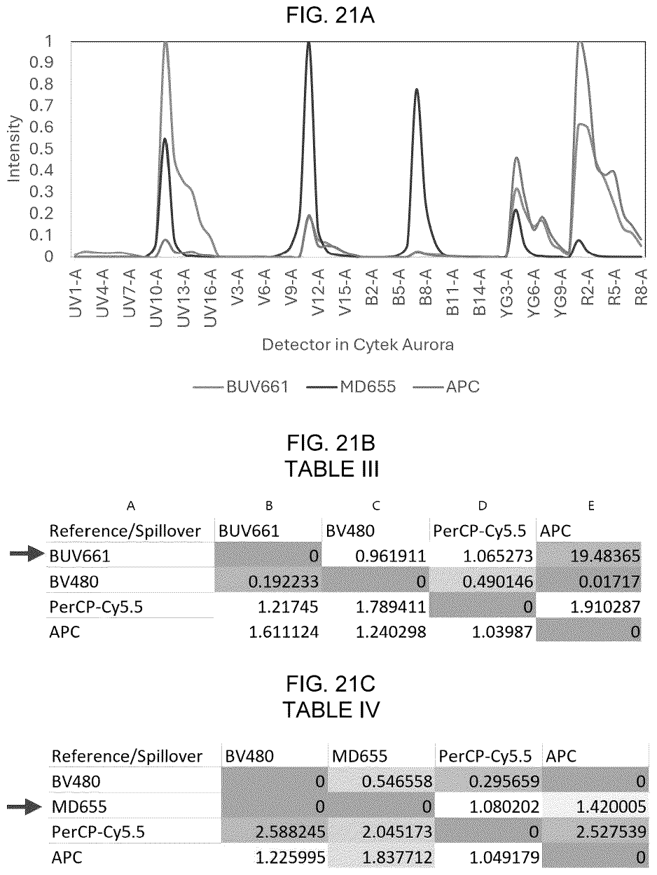

Now, with primary reference to FIGS. 21A, 21B—Table III and 21C—Table IV which provides a SSM comparison including fluorophores selected from the optimized multicolor immunofluorescence panel (OMIP) in combination (as shown in the example of Table III) and in substitution with MultiDot 665 (as shown in the example of Table IV) to demonstrate the reduction in the allophycocyanin (“APC”) channel by substitution of MultiDot 655 (“MD655”) for the Brilliant Utraviolet 661 (“BUV661”) fluorophore.

CytoCompcells® obtained from Beckman Coulter were resuspended to 1 ml with provided serum. Cells were pipetted up and down to create a single cell suspension. To the resuspended cells, 10 ml of PBS buffer+2 mM EDTA+5% BSA (“FACS buffer”) was added, and cells were centrifuged at 1800 RPMs for 7 minutes. Serum and buffer were aspirated off the cell pellet and cells were resuspended in 900 μl FACS buffer. Aliquots in the amount of 100 μl were taken from the washed resuspended cells and placed into 9 flow cytometry tubes (12×75 mm) and labeled as unlabeled, BUV661, BV480, PerCP-Cy5.5, and APC in the first data set. In the second data set tubes were labeled as BV480, PerCP-Cy5.5, APC and MultiDot 655 to replace the APC label. To each of the tubes except for the unlabeled control, 5 μl of anti-CD4 antibody was added to the appropriately labeled tube. In the MD655 (MultiDots) 10 μl of Multidots conjugated to the CD4 antibody was added. Cells with labels were incubated at room temperature for 30 minutes and then washed with 2× the staining volume with FACS buffer. The supernatant was aspirated and the cells were resuspended in 250 μl of FACS buffer per tube and immediately run on the Cytek Aurora spectral flow cytometer to determine spillover spread matrix and how MD655 affects other colors versus APC.

Single stain BUV661 and MD655-Ab CD4 when visualized in APC filter shows that MD655-Ab CD4 spreads very minimally into the APC filter having a value of 1.4 as compared to BUV661 having a value of 19.48.

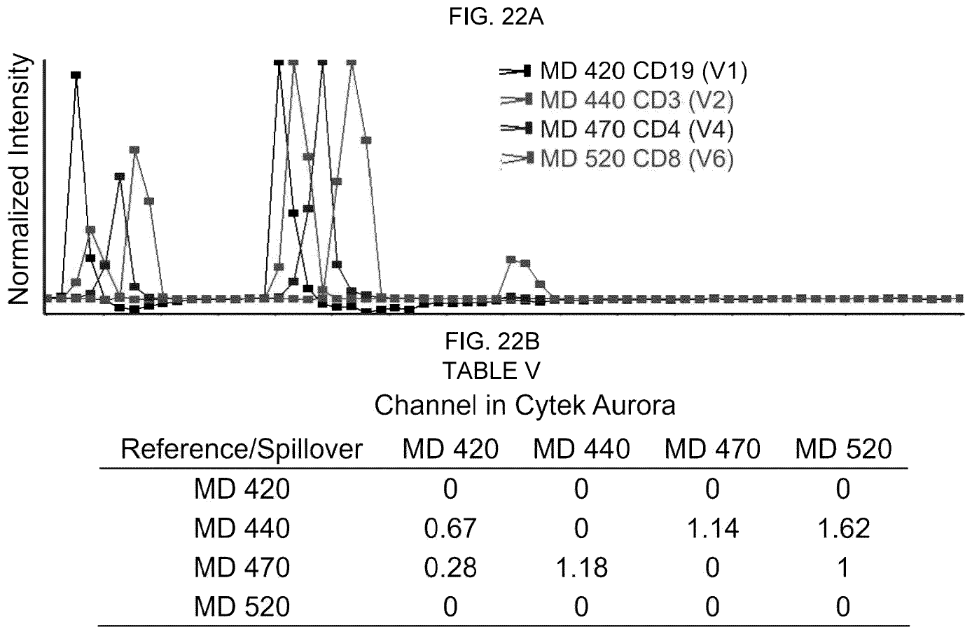

Now with primary reference to FIG. 22A and Table V, which provides a histogram obtained by analysis of a multiplex PNEP-MultiDot using a CYTEK AURORA® spectral flow cytometry available from Cytek Biosciences, Fremont, California and a SSM comparison of the results.

Lyophilized lymphocytes were labelled with MultiDot 420-Ab CD19, MultiDot 440-Ab CD3, MultiDot 470-Ab CD4 and MultiDot 520-Ab CD8. The multiplex sample was analyzed with the signal distinctly captured in channels V1, V2, V4 and V6 without significant spread in adjacent channels and without affecting the SSM as shown in the example of Table V.

The results evidence that multiplexing can be achieved using PNEP-MultDots-Ab CD labeled cells including QDs (2a) of four to five colors along the visible spectrum wavelength of 420 nm to 520 nm spanning 100 nm without affecting the SSM (as shown in the example of Table V). Additionally, this can be achieved with a single laser either deep UV, UV or violet.

As can be easily understood from the foregoing, the basic concepts of the present invention may be embodied in a variety of ways. The invention involves numerous and varied embodiments of polymer encapsulated nanoparticles and methods for making and using such a polymer encapsulated nanoparticles.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “encapsulated nanoparticle” should be understood to encompass disclosure of the act of “encapsulating a nanoparticle”—whether explicitly discussed or not—and, conversely, were there effectively disclosure of the act of “encapsulating a nanoparticle”, such a disclosure should be understood to encompass disclosure of a “encapsulated nanoparticle” and even a “means for encapsulating a nanoparticle.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Thus, the applicant(s) should be understood to claim at least: i) each of the polymer encapsulated nanoparticles herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The elements following an open transitional phrase such as “comprising” may in the alternative be claimed with a closed transitional phrase such as “consisting essentially of” or “consisting of” whether or not explicitly indicated the description portion of the specification.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

We claim:

1. A nanocomposite, comprising:
an amphiphile having a hydrophobic region and a hydrophilic region; and
at least one quantum dot encapsulated by association with said hydrophobic region of said amphiphile;
wherein said at least one quantum dot selected from a quantum dot set, wherein each quantum dot within said quantum dot set having a peak emission separated by twenty nanometer wavelengths between 420 nm to 680 nm or having peak emission separated by fifty nanometer wavelengths between 700 nm to 1000 nm.

2. The nanocomposite of claim 1, wherein said quantum dot set includes quantum dots having peak emissions at 420 nm, 440 nm, 460 nm, 480 nm, 500 nm, 520 nm, 540 nm, 560 nm, 580 nm, 600 nm, 620 nm, 640 nm, 660 nm and 680.

3. The nanocomposite of claim 1, wherein said quantum dot set includes quantum dots having peak emissions at 410 nm, 430 nm, 450 nm, 470 nm, 490 nm, 510 nm, 530 nm, 550 nm, 570 nm, 590 nm, 610 nm, 630 nm, 650 nm and 670.

4. The nanocomposite of claim 1, wherein said quantum dot set includes four or five quantum dots each having a peak emission within a 100 nm wavelength occurring within 420 nm to 680 nm.

5. The nanocomposite of claim 4, wherein said 100 nm wavelength is selected from the group of 320 nm to 420 nm, 340 nm to 440 nm, 360 nm to 460 nm, 380 nm to 480 nm, 400 nm to 500 nm, 420 nm to 520 nm, 440 nm to 540, 460 nm to 560 nm, 480 nm to 580 nm, 500 nm to 600 nm, 520 nm to 620, 540 nm to 640 nm, 560 nm to 660 nm, and 580 nm to 680 nm.

6. The nanocomposite of claim 1, wherein said quantum dot set includes quantum dots having peak emissions at 700 nm, 750 nm, 800 nm, 850 nm, 900 nm, 950 nm, and 1000 nm.

7. The nanocomposite of claim 1, wherein said quantum dot set includes two to four quantum dots each having a peak emission within a 100 nm wavelength occurring within 700 nm to 1000 nm.

8. The nanocomposite of claim 1, wherein said 100 nm wavelength is selected from the group of 700 nm to 800 nm, 750 nm to 850 nm, 800 nm to 900 nm, 850 nm to 950 nm and 900 nm to 1000 nm.

9. The nanocomposite of any one of claim 4 or 7, wherein upon capture of said peak emissions by spectral flow cytometry within said 100 nm wavelength, any pair of said nanocomposites within said mixture of nanocomposites from within said set of nanocomposites has a spectral flow cytometry similarity index of less than 0.9.

10. The nanocomposite of claim 1, wherein said amphiphile includes a functional group.

11. The nanocomposite of claim 10, further comprising a linker that binds to said functional group.

12. The nanocomposite of claim 10, further comprising an antibody or an antibody fragment bound to said functional group.

13. The nanocomposite of claim 11, further comprising an antibody or an antibody fragment bound to said functional group.

14. The nanocomposite of any one of claim 12 or 13, further comprising a cellular target bound by said antibody or said antibody fragment.

15. The nanocomposite of claim 14, wherein said antibody or said antibody fragment specifically binds a cellular target.

16. The nanocomposite of claim 15, wherein said antibody or antibody fragment which binds a peripheral blood mononuclear cell.

17. The nanocomposite of claim 16, wherein said peripheral blood mononuclear cell includes cluster of differentiation marker (CD marker).

18. The nanocomposite of claim 17, wherein said CD marker is selected from the group of: CD3, CD4, CD8, CD14, CD19, CD 20, CD22, and CD34.

19. The nanocomposite of claim 15, wherein said nanocomposite bound to said antibody or antibody fragment binding said cellular target analyzed by spectral flow cytometry to detect said cellular target based on the spectral signature of said at least one quantum dot selected from within said set of quantum dots.

20. The nanocomposite of claim 1 wherein said amphiphile comprises a polystyrene-b-polyethylene glycol.

21. The nanocomposite of claim 20, wherein said polystyrene-b-polyethylene glycol a branched polyethylene glycol.

22. The nanocomposite of claim 21, wherein said is selected from the group of: a 4-arm polyethylene glycol, a six arm polyethylene glycol, and an 8-arm polyethylene glycol.

23. A nanocomposite, comprising:
an amphiphile having a hydrophobic region and a hydrophilic region; and
at least one quantum dot encapsulated by association with said hydrophobic region of said amphiphile;
wherein said at least one quantum dot selected from a quantum dot set, wherein said quantum dot set includes four or more quantum dots each having a peak emission within a 100 nm wavelength occurring within 420 nm to 680 nm or includes at least two to four quantum dots each having peak emission with a 100 nm wavelength occurring within 700 nm to 1000 nm, wherein in a multiplex sample each peak emission within said 100 nm wavelength having an individual spectral signature resolved by spectral flow cytometry.

24. The nanocomposite of claim 23, wherein any pair of said nanocomposites within said mixture of nanocomposites from within said set of nanocomposites has a spectral flow cytometry similarity index of less than 0.9.

25. The nanocomposite of claim 23, wherein said quantum dot set includes quantum dots having a peak emissions separated by twenty nanometer wavelengths between 420 nm to 680 nm or having peak emissions separated by fifty nanometers between 700 nm to 1000 nm.

26. The nanocomposite of claim 25, wherein said quantum dot set includes quantum dots having peak emissions peak emissions at 420 nm, 440 nm, 460 nm, 480 nm, 500 nm, 520 nm, 540 nm, 560 nm, 580 nm, 600 nm, 620 nm, 640 nm, 660 nm and 680.

27. The nanocomposite of claim 1, wherein said quantum dot set includes quantum dots having peak emissions at 700 nm, 750 nm, 800 nm, 850 nm, 900 nm, 950 nm, and 1000 nm.

28. The nanocomposite of claim 1, wherein said amphiphile includes a functional group.

29. The nanocomposite of claim 28, further comprising a linker that binds to said functional group.

30. The nanocomposite of claim 28, further comprising an antibody or an antibody fragment bound to said functional group.

31. The nanocomposite of claim 29, further comprising an antibody or an antibody fragment bound to said functional group.

32. The nanocomposite of any one of claim 30 or 31, further comprising a cellular target bound by said antibody or said antibody fragment.

33. The nanocomposite of claim 32, wherein said antibody or said antibody fragment specifically binds a cellular target.

34. The nanocomposite of claim 33, wherein said antibody or antibody fragment which binds a peripheral blood mononuclear cell.

35. The nanocomposite of claim 34, wherein said peripheral blood mononuclear cell includes cluster of differentiation marker (CD marker).

36. The nanocomposite of claim 35, wherein said CD marker is selected from the group of: CD3, CD4, CD8, CD14, CD19, CD 20, CD22, and CD34.

37. The nanocomposite of claim 33, wherein said nanocomposite bound to said antibody or antibody fragment binding said cellular target analyzed by spectral flow cytometry to detect said cellular target based on the spectral signature of said at least one quantum dot selected from within said set of quantum dots.

38. The nanocomposite of claim 1 wherein said amphiphile comprises a polystyrene-b-polyethylene glycol.

39. The nanocomposite of claim 38, wherein said polystyrene-b-polyethylene glycol a branched polyethylene glycol.

40. The nanocomposite of claim 39, wherein said is selected from the group of: a 4-arm polyethylene glycol, a six arm polyethylene glycol, and an 8-arm polyethylene glycol.

41. A quantum dot set, comprising a plurality of quantum dots, wherein each quantum dot within said quantum dot set having a peak emission separated by twenty nanometer wavelengths between 420 nm to 680 nm and having a peak emission separated by fifty nanometer wavelengths between 700 nm to 1000 nm.

42. The quantum dot set of claim 41, wherein said quantum dot set includes quantum dots having peak emissions at 420 nm, 440 nm, 460 nm, 480 nm, 500 nm, 520 nm, 540 nm, 560 nm, 580 nm, 600 nm, 620 nm, 640 nm, 660 nm and 680.

43. The quantum dot set of claim 41, wherein in a multiplex sample each peak emission within having an individual spectral signature resolved by spectral flow cytometry.

44. The quantum dot set of claim 43, wherein any pair of said quantum dots within quantum dot set has a spectral flow cytometry similarity index of less than 0.9.

45. A quantum dot set, comprising a plurality of quantum dots, wherein each quantum dot within said quantum dot set having a peak emission separated by fifty nanometer wavelengths between 400 nm to 1000 nm.

46. The quantum dot set of claim 41, wherein said quantum dot set including quantum dots having a peak emissions at 700 nm, 750 nm, 800 nm, 850 nm, 900 nm, 950 nm and 1000 nm.

47. The quantum dot set of claim 46, wherein in a multiplex sample each peak emission within having an individual spectral signature resolved by spectral flow cytometry.

48. The quantum dot set of claim 46, wherein any pair of said quantum dots within quantum dot set has a spectral flow cytometry similarity index of less than 0.9.
